(19) United States
(12) Patent Application Publication
(10) Pub. No.: US 2006/0040861 A1
(43) Pub. Date: Feb. 23, 2006

(54) Method of treating, preventing, and diagnosing prostate cancer

(76) Inventor: Yoshiki Iwamoto, Arcadia, CA (US)

Correspondence Address: QUARLES & BRADY LLP, 411 E. WISCONSIN AVENUE, SUITE 2040, MILWAUKEE, WI, 53202-4497, US

(21) Appl. No.: 11/191,433

(22) Filed: Jul. 28, 2005

Related U.S. Application Data
(60) Provisional application No. 60/652,165, filed on Feb. 7, 2005.
(60) Provisional application No. 60/592,204, filed on Jul. 29, 2004.
(60) Provisional application No. 60/607,029, filed on Sep. 3, 2004.

(51) Int. Cl. A61K 38/17; A61K 48/00

(52) U.S. Cl. 514/12; 514/44

(57) ABSTRACT

A method of treating prostate cancer in a prostate cancer patient is disclosed. In one embodiment of the present invention, the method comprises the step of decreasing or blocking the patient's leptin interaction with leptin receptor or increasing adiponectin interaction with adiponectin receptors.

### CROSS-REFERENCE TO RELATED APPLICATIONS

This application claims priority to U.S. provisional 60/652,165, filed Feb. 7, 2005; 60/592,204, filed Jul. 29, 2004; and 60/607,029, filed Sep. 3, 2004. All applications are incorporated by reference within as if set forth fully.

### STATEMENT REGARDING FEDERALLY SPONSORED RESEARCH OR DEVELOPMENT

### BACKGROUND OF THE INVENTION

Prostate cancer is the most common male malignancy and the second most common cause of cancer-related death among men in the United States. The disease process is characterized by a prolonged natural history. Despite its relatively slow growth, a number of patients have persistent and/or recurrent disease. Initial treatment for many patients with recurrent disease is hormonal therapy to remove or decrease serum androgen as a potential growth stimulant for the prostate cancer. While this approach is initially effective in the majority of patients, ultimately the disease becomes resistant to the loss of androgen, returns and, in many cases, culminates in the death of the patient. The molecular mechanism of this “hormone resistance” needs to be clarified to develop effective strategies to prevent and treat hormone-resistant prostate cancer.

Several lines of evidence indicate that obesity (adiposity) is associated with prostate cancer risk, particularly with clinical features characteristics of the accelerated progression of prostate cancer (Amling, C. L., et al., Urology 58:723-728, 2001; Furuya, Y., et al., Int. J. Urol. 5:134-137, 1998; Hsing, A. W., et al., Cancer Epidemiol. Biomarkers Prev. 9:1335-1341, 2000; Rodriguez, C., et al., Cancer Epidemiol. Biomarkers Prev. 10:345-353, 2001). However, little is known about the molecular mechanism of this association.

The present invention provides methods of treating, preventing, and diagnosing prostate cancer based on our findings that adipose factors play a crucial role in prostate cancer cell growth, including androgen-independent cell growth.

### SUMMARY OF THE INVENTION

In one embodiment, the present invention is a method of treating prostate cancer in a prostate cancer patient, comprising the step of decreasing or blocking the patient's leptin function or increasing adiponectin function. In a preferred embodiment, the method additionally comprises the step of decreasing or blocking the patient's IGF or IL-6 function.

In another embodiment, the present invention is a method of diagnosing a patient's prostate cancer risk comprising the step of examining the patient's blood concentration profiles of adipose cytokines in combination and correlating the level with prostate cancer diagnosis. The adipose cytokines are preferably selected from the group consisting of leptin, adiponectin IGF-I, IGF-II, IL-6, and TNF-α. Preferably, one also combines PSA and DHT.

### BRIEF DESCRIPTION OF THE SEVERAL VIEWS OF THE DRAWINGS

FIG. 1 describes [³H] thymidine incorporation by prostate cancer cells in response to the conditioned medium from human adipocyte culture. FIG. 1A: Preadipocytes (left panel) were differentiated into mature adipocytes (right panel) after the treatment with 10 μg/ml insulin, 500 μM isobutyl-methylxanthine, 1 μM dexamethasone, and 200 μM indomethacin for 10 days. Cells were stained with Oil-Red-O to visualize fat droplets and photographed at X 200 magnification under a bright-field microscope. To prepare conditioned media, preadipocytes and mature adipocytes were cultured in the serum-free medium for 24 hours. Cell culture supernatants were harvested, stored at −80° C. and used as conditioned media. FIG. 1B: DU145, PC-3 and LNCaP-FGC cells were deprived of serum for 48 hours, and incubated with the control, serum-free medium, preadipocyte conditioned medium or mature adipocyte conditioned medium for 20 hours. [³H] thymidine incorporation was measured during the last 5 hours. Values represent the mean±SD of quadruplicate samples of a representative experiment.

FIG. 2 illustrates expression profiles of leptin receptor isoforms in prostate cancer cells. The quantitative RT-PCR analysis profiled the mRNA expression of leptin receptor isoforms in DU145, PC-3 and LNCaP-FGC cells. Leptin receptor has 4 isoforms (huOB-R, huB219.1-3). After cells were deprived of serum for 24 hours, total RNA was prepared and subjected to quantitative RT-PCR analysis. Amplifications were performed with 33, 36, 39 cycles for huOB-R, huB219.1, huB219.2 and huB219.3, or 19, 22, 25 cycles for GAPDH.

FIG. 3 is a set of bar graphs illustrating leptin stimulating cell proliferation in androgen-independent DU145 and PC-3 prostate cancer cells but not in androgen-dependent LNCaP-FGC cells. FIG. 3A: DU145, PC-3 and LNCaP-FGC cells were serum-deprived for 24 hours and stimulated with the indicated concentrations of leptin for 20 hours. [³H] thymidine incorporation was measured during the last 5 hours. Values represent the mean±SD of quadruplicate samples of a representative experiment. FIG. 3B: DU145, PC-3 and LNCaP-FGC cells were deprived of serum for 24 hours and incubated with or without 12.5 μg/ml leptin for 5 days. Cell viability was measured by the enzymatic reduction of MTT (O.D. 550 nm -670 nm) during the last 3 hours. Values represent the mean±SD of quadruplicate samples of a representative experiment.

FIG. 4 is a set of Western blots illustrating that leptin activates JNK in androgen-independent DU145 and PC-3 prostate cancer cells but not in androgen-dependent LNCaP-FGC cells. Androgen-independent DU145 and PC-3 prostate cancer cells and androgen-dependent LNCaP-FGC cells were deprived of serum for 24 hours and incubated in serum-free medium (lanes 1, 9 and 17), or the serum-free medium containing 12.5 μg/ml leptin (lanes 2-7,10-15 and 18-23) or 10 μg/ml anisomycin (Ani) (lane 8, 16 and 24) for indicated periods. Anisomycin served as a positive control to stimulate JNK activation. Cell lysates (250 μg protein) were subjected to the in vitro JNK assay with N-terminal c-Jun fusion protein as a substrate. Phosphorylation of the substrate protein on Ser 63 was detected by Western blot analysis using the specific antibody (phospho-N-terminal c-Jun fusion protein (Ser63)). To normalize JNK activity to total JNK protein levels, cell lysates (100 μg protein) were applied to Western blot analysis using the anti-JNK antibody that detects both active and inactive forms of JNK (p54 JNK and p46 JNK).

FIG. 5 is a set of Western blots illustrating that leptin stimulates phosphorylation of c-Jun, an endogenous JNK substrate, during androgen-independent prostate cancer cell proliferation. Androgen-independent DU145 and PC-3 prostate cancer cells were serum-starved for 24 hours and treated with either 12.5 μg/ml leptin (lanes 2-7 and 10-15) or 10 μg/ml anisomycin (Ani) (lanes 8 and 16) for 15 minutes. Untreated (lanes 1 and 9) and anisomycin-treated cells served as positive and negative controls. Cell lysates (100 μg protein) were subjected to Western blot analysis. c-Jun phosphorylation at Ser-63 and Ser-73 was determined with phospho-c-Jun (Ser-63) and (Ser-73) antibodies (phospho-c-Jun (Ser-63) and phospho-c-Jun (Ser-73)). To normalize c-Jun phosphorylation levels to total amounts of c-Jun protein, membranes probed with these antibodies were stripped and re-probed with the anti-c-Jun antibody that recognizes both phosphorylated and non-phosphorylated forms of c-Jun (c-Jun).

FIG. 6 demonstrates that JNK activation is required for leptin-mediated, androgen-independent prostate cancer cell proliferation. FIG. 6A: Androgen-independent DU145 and PC-3 prostate cancer cells were deprived of serum for 24 hours and treated with 12.5 μg/ml leptin for 15 minutes, with (lanes 4-8 and 12-16) or without (lanes 3 and 11) pretreatment with SP600125, a JNK inhibitor, for 30 minutes at indicated concentrations. Cells without any treatment (lanes 1 and 9) and treated with DMSO alone (lanes 2 and 10) were included as controls. Phosphorylation of c-Jun on Ser-63 and Ser-73 residues was assessed by Western blot analysis with phospho-c-Jun (Ser-63) and (Ser-73) antibodies (phospho-c-Jun (Ser-63) and phospho-c-Jun (Ser-73)). To normalize c-Jun phosphorylation levels to total amounts of c-Jun protein, membranes were then stripped and re-probed with the antibody that recognizes both phosphorylated and non-phosphorylated form of c-Jun (c-Jun). FIG. 6B: After a 24-hour serum deprivation, DU145 and PC-3 cells were pretreated with SP600125 at indicated concentrations for 30 minutes, followed by leptin stimulation (12.5 μg/ml) for 20 hours. Cell proliferation was measured by [³H] thymidine incorporation during the last 5 hours. Values represent the mean±SD of quadruplicate samples of a representative experiment.

FIG. 7 is a set of bar graphs demonstrating interaction of leptin with IGF-I and IL-6 in androgen-independent prostate cancer cell proliferation. DU145, PC-3, and LNCaP-FGC cells were serum-deprived for 48 hours and stimulated with 12.5 μg/ml leptin in combination with 100 ng/ml IL-6 (FIG. 7A) or 100 ng/ml IGF-1 (FIG. 7B) for 20 hours. Cell proliferation was measured by [³H] thymidine incorporation during the last 5 hours. Values represent the mean±SD of quadruplicate samples of a representative experiment.

FIG. 8 is an expression profile of adiponectin receptor 1 and 2 in prostate cancer and hepatocellular carcinoma cells. Quantitative reverse transcriptase-PCR analysis profiled the mRNA expression of adiponectin receptor isoforms in prostate cancer DU145, PC-3 and LNCaP-FGC cells, and hepatocellular carcinoma HepG2 cells. Adiponectin receptor has two isoforms (adiponectin receptor 1 and 2). After cells were deprived of serum for 24 hours, total RNA was prepared and subjected to quantitative reverse transcriptase-PCR analysis. Amplifications were performed with 23, 26, and 29 cycles for adiponectin receptor 1 (AdipoR1) and adiponectin receptor 2 (AdipoR2) or 18, 21, and 24 cycles for GAPDH.

FIG. 9A demonstrates that f-adiponectin activates JNK in prostate cancer cells, hepatocellular carcinoma cells, and myoblasts. Prostate cancer DU145, PC-3 and LNCaP-FGC cells, hepatocellular carcinoma HepG2 cells, and C2C12 myoblasts were deprived of serum for 24 hours and incubated in serum-free medium (lanes 1, 9, 17, 25, and 33) or the serum-free medium containing 1.0 μg/ml f-adiponectin (lanes 2-7, 10-15, 18-23, 26-31, and 34-39) or 10 μg/ml anisomycin (Anis) (lanes 8, 16, 24, 32, and 40) for indicated periods. Anisomycin served as a positive control to stimulate JNK activation. Cell lysates (250-500 μg protein) were subjected to the in vitro JNK assay with N-terminal c-Jun fusion protein as a substrate. Phosphorylation of the substrate protein on Ser-63 was detected by Western blot analysis using the specific antibody (phospho-N-terminal c-Jun fusion protein (Ser-63)). To normalize JNK activity to total JNK protein levels, cell lysates (100 μg protein) were applied to Western blot analysis using the anti-JNK antibody that detects both active and inactive forms of JNK (p54 JNK and p46 JNK).

FIG. 9B demonstrates that g-adiponectin activates JNK in prostate cancer cells, hepatocellular carcinoma cells, and myoblasts. Prostate cancer DU145, PC-3 and LNCaP-FGC cells, hepatocellular carcinoma HepG2 cells, and C2C12 myoblasts were deprived of serum for 24 hours and incubated in serum-free medium (lanes 1, 9, 17, 25, and 33) or the serum-free medium containing 1.0 μg/ml g-adiponectin (lanes 2-7, 10-15, 18-23, 26-31, and 34-39) or 10 μg/ml anisomycin (Anis) (lanes 8, 16, 24, 32, and 40) for indicated periods. Anisomycin served as a positive control to stimulate JNK activation. Cell lysates (250-500 μg protein) were subjected to the in vitro JNK assay with N-terminal c-Jun fusion protein as a substrate. Phosphorylation of the substrate protein on Ser-63 was detected by Western blot analysis using the specific antibody (phospho-N-terminal c-Jun fusion protein (Ser-63)). To normalize JNK activity to total JNK protein levels, cell lysates (100 μg protein) were applied to Western blot analysis using the anti-JNK antibody that detects both active and inactive forms of JNK (p54 JNK and p46 JNK).

FIG. 10A illustrates that f-adiponectin stimulates phosphorylation of c-Jun, an endogenous JNK substrate, in prostate cancer cells, hepatocellular carcinoma cells, and myoblasts. Prostate cancer DU145, PC-3 and LNCaP-FGC cells, hepatocellular carcinoma HepG2 cells, and C2C12 myoblasts were serum-starved for 24 hours and treated with either 1.0 μg/ml f-adiponectin (lanes 2-7, 10-15, 18-23, 26-31, and 34-39) or 10 μg/ml anisomycin (Anis) (lanes 8, 16, 24, 32, and 40) for 15 minutes. Untreated (lanes 1, 9, 17, 25, and 33) and anisomycin-treated cells served as negative and positive controls. Cell lysates (100 μg protein) were subjected to Western blot analysis. c-Jun phosphorylation at Ser-63 and Ser-73 was determined with phospho-c-Jun (Ser-63) and (Ser-73) antibodies (phospho-c-Jun (Ser-63) and phospho-c-Jun (Ser-73)). To normalize c-Jun phosphorylation levels to total amounts of c-Jun protein, membranes probed with these antibodies were stripped and re-probed with the anti-c-Jun antibody that recognizes both phosphorylated and non-phosphorylated forms of c-Jun (c-Jun).

FIG. 10B illustrates that g-adiponectin stimulates phosphorylation of c-Jun, an endogenous JNK substrate, in prostate cancer cells, hepatocellular carcinoma cells, and myoblasts. Prostate cancer DU145, PC-3 and LNCaP-FGC cells, hepatocellular carcinoma HepG2 cells, and C2C12 myoblasts were serum-starved for 24 hours and treated with either 1.0 μg/ml g-adiponectin (lanes 2-7, 10-15, 18-23, 26-31, and 34-39) or 10 μg/ml anisomycin (Anis) (lanes 8, 16, 24, 32, and 40) for 15 minutes. Untreated (lanes 1, 9, 17, 25, and 33) and anisomycin-treated cells served as negative and positive controls. Cell lysates (100 μg protein) were subjected to Western blot analysis. c-Jun phosphorylation at Ser-63 and Ser-73 was determined with phospho-c-Jun (Ser-63) and (Ser-73) antibodies (phospho-c-Jun (Ser-63) and phospho-c-Jun (Ser-73)). To normalize c-Jun phosphorylation levels to total amounts of c-Jun protein, membranes probed with these antibodies were stripped and re-probed with the anti-c-Jun antibody that recognizes both phosphorylated and non-phosphorylated forms of c-Jun (c-Jun).

FIG. 11A demonstrates that STAT3 is constitutively activated in DU145 and HepG2 cells. Prostate cancer DU145 cells and hepatocellular carcinoma HepG2 cells were serum-starved for 24 hours, and cell lysates were prepared. Cell lysates (10 μg protein) were subjected to the electromobility shift assay using ³²P-end-labeled M67-SIE as a probe. The STAT3-DNA complex (STAT3) was observed and supershifted (SS) by anti-STAT3 antibody.

FIG. 11B demonstrates that f-adiponectin inhibits STAT3 DNA binding activity in prostate cancer DU145 cells and hepatocellular carcinoma HepG2 cells. Prostate cancer DU145 cells and hepatocellular carcinoma HepG2 cells were deprived of serum for 24 hours and incubated in serum-free medium (lanes 1 and 8) or serum-free medium containing 1.0 μg/ml f-adiponectin (lanes 2-7 and 9-14) for various periods up to 60 minutes. Cell lysates (10 μg protein) were subjected to the electromobility shift assay using ³²P-end-labeled M67-SIE as a probe. STAT3, STAT3-DNA complex.

FIG. 11C demonstrates that g-adiponectin inhibits STAT3 DNA binding activity in prostate cancer DU145 cells and hepatocellular carcinoma HepG2 cells. Prostate cancer DU145 cells and hepatocellular carcinoma HepG2 cells were deprived of serum for 24 hours and incubated in serum-free medium (lanes 1 and 8) or serum-free medium containing 1.0 μg/ml g-adiponectin (lanes 2-7 and 9-14) for various periods up to 60 minutes. Cell lysates (10 μg protein) were subjected to the electromobility shift assay using ³²P-end-labeled M67-SIE as a probe. STAT3, STAT3-DNA complex.

FIG. 12 is a set of bar graphs demonstrating that f-adiponectin, but not g-adiponectin, inhibits prostate cancer cell growth at subphysiological concentrations. DU145, PC-3, and LNCaP-FGC cells were serum-deprived for 24 hours and treated with indicated concentrations of f-adiponectin (FIG. 12A) or g-adiponectin (FIG. 12B) for 5 days. Cell viability was measured by the enzymatic reduction of MTT (OD 550-670 nm) during the last 3 hours. Values represent the mean±S.D. of quadruplicate samples of a representative experiment. Upper panels demonstrate results of adiponectin treatment at low concentrations up to 1 μg/ml (Low), and lower panels show those at high concentrations between 1 and 30 μg/ml (High). G, g-adiponectin; F, f-adiponectin.

FIG. 13 demonstrates that the HMW form, but not lower molecular weight forms, of f-adiponectin is inhibitory to prostate cancer cell growth. FIG. 13A: Fractions produced by velocity sedimentation of an f-adiponectin lot were subjected to Western blot analysis with anti-adiponectin antibody. Velocity sedimentation separated high molecular weight (HMW) and lower molecular weight (LMW: trimer and hexamer) forms of f-adiponectin. FIG. 13B: Serum-deprived DU145 cells were treated with the same fractions (solid bars) and subjected to the MTT assay to determine the effect of different oligomeric forms of f-adiponectin on cell growth. Corresponding fractions from f-adiponectin-free velocity sedimentation were employed as controls (open bars). f-Adiponectin (1 μg/ml) without velocity sedimentation was used as a positive control for the MTT assay. Values represent the mean±S.D. of triplicate samples of a representative experiment.

FIG. 14 is a set of bar graphs demonstrating that f-adiponectin inhibits adipocyte conditioned medium-induced, prostate cancer cell growth. DU145, PC-3, and LNCaP-FGC cells were serum-starved for 24 hours and treated for 5 days with the control serum-free medium, preadipocyte conditioned medium, or mature adipocyte conditioned medium in the absence (open bars) or presence (solid bars) of 1 μg/ml f-adiponectin. Cell viability was measured by the enzymatic reduction of MTT (OD 550-670 nm) during the last 3 hours. Values represent the mean±S.D. of quadruplicate samples of a representative experiment.

FIG. 15 demonstrates that f-adiponectin inhibits leptin- and/or IGF-I-stimulated, androgen-independent prostate cancer cell growth. Androgen-independent DU145 cells were deprived of serum for 24 hours and treated for 5 days with 12.5 μg/ml leptin and/or 100 ng/ml IGF-I in the absence (open bars) or presence (solid bars) of 1 μg/ml f-adiponectin. Cell viability was measured by the enzymatic reduction of MTT (OD 550-670 nm) during the last 3 hours. Values represent the mean±S.D. of quadruplicate samples of a representative experiment.

FIG. 16 demonstrates that f-adiponectin inhibits DHT-stimulated, androgen-dependent prostate cancer cell growth. Androgen-dependent LNCaP-FGC cells were deprived of serum for 24 hours and treated for 5 days with 10 nM DHT in the absence (open bars) or presence (solid bars) of 1 μg/ml f-adiponectin. Cell viability was measured by the enzymatic reduction of MTT (OD 550-670 nm) during the last 3 hours. Values represent the mean±S.D. of quadruplicate samples of a representative experiment.

FIG. 17 demonstrates that f-adiponectin enhances doxorubicin suppression of prostate cancer cell growth. After 24-hour serum deprivation, DU145, PC-3, and LNCaP-FGC cells were treated for 5 days with doxorubicin at concentrations up to 1.92 μg/ml in the absence (open bars) or presence (solid bars) of 1 μg/ml f-adiponectin. Cell viability was measured by the enzymatic reduction of MTT (OD 550-570 nm) during the last 3 hours. Values represent the mean±S.D. of quadruplicate samples of a representative experiment.

### DETAILED DESCRIPTION OF THE INVENTION

A. In General

Prostate Cancer Cell Growth Stimulation by Adipose Cytokines: Adipose Cytokines as Targets for Novel Therapeutic, Preventive and Diagnostic Strategies for Prostate Cancer

The following summarizes some of our findings described in Example A, below:
i. Adipose factors stimulate prostate cancer cell proliferation both in androgen-independent and androgen-dependent prostate cancer cells.
ii. Prostate cancer cells express functional receptors for leptin, one of the major adipose cytokines that controls body weight homeostasis through food intake and energy expenditure. Furthermore, leptin stimulates androgen-independent prostate cancer cell proliferation.
iii. c-Jun NH₂-terminal kinase (JNK), a Ser/Thr kinase playing a crucial role in obesity and type II diabetes mellitus, mediates leptin-stimulated, androgen-independent prostate cancer cell proliferation through c-Jun phosphorylation (activation). Pharmacological JNK inhibition blocks leptin-stimulated, androgen-independent prostate cancer cell proliferation, as well as c-Jun phosphorylation.
iv. Leptin interacts with other adipose cytokines, insulin-like growth factor-I (IGF-I) and interleukin-6 (IL-6), to stimulate androgen-independent prostate cancer cell proliferation.

In addition to the findings described in Example A, we discovered the following:
v. Leptin stimulates androgen-dependent prostate cancer cell proliferation. Leptin and androgen (dihydrotestosterone (DHT)) synergistically stimulate cell proliferation in androgen-dependent prostate cancer LNCaP-FGC cells. Leptin stimulates cell proliferation in androgen-dependent prostate cancer cells only in the presence of DHT but not in the absence of DHT. This indicates that leptin is crucial in androgen-dependent prostate cancer cell growth, as well as androgen-independent prostate cancer cell growth.
vi. Prostate cancer cells produce leptin. This suggests that leptin, as well as IGF-I (Pietrzkowski, Z., et al., Cancer Res. 53:1102-1106, 1993), IGF-II (Angelloz-Nicoud, P., and M. Binoux, Endocrinology 136:5485-5492, 1995; Figueroa, J. A., et al., J. Clin. Endocrinol. Metab. 80:3476-3482, 1995) and IL-6 (Okamoto, M., et al., Cancer Res. 57:141-146, 1997), can stimulate prostate cancer cell growth in an autocrine, as well as a paracrine, manner.
vii. Adiponectin is another adipose cytokine and has been shown to be important in obesity and type II diabetes mellitus (Comuzzie, A. G., et al., J. Clin. Endocrinol. Metab. 86:4321-5, 2001; Kissebah, A. H., et al., Proc. Natl. Acad. Sci. USA 97:14478-83, 2000; Maeda, N., et al., Nat. Med. 8:731-7, 2002; Yamauchi, T., et al., Nat. Med. 7:941-6, 2001). Full-length adiponectin (f-adinponectin) inhibits androgen-independent and -dependent prostate cancer cell growth remarkably.
viii. Pharmacological inhibition of JNK with SP600125, a pharmacological JNK inhibitor, blocks leptin-induced, cell cycle progression in the G₁ phase of cell cycle.
ix. Pharmacological inhibition of JNK with SP600125, a pharmacological JNK inhibitor, induces apoptosis in androgen-independent prostate cancer cells.
x. Phosphatidylinositol-3 (P13) kinase mediates leptin- and/or IGF-stimulated, androgen-independent prostate cancer cell proliferation via AKT activation. P13 kinase inhibition with LY294002, a pharmacological P13 kinase inhibitor, suppresses leptin- and/or IGF-stimulated, androgen-independent prostate cancer cell proliferation, as well as leptin and/or IGF-induced, AKT phosphorylation (activation).

Our data demonstrate that leptin, a major adipose cytokine, stimulates prostate cancer cell proliferation. Furthermore, IGF-I (Pietrzkowski, Z., et al., supra, 1993), IGF-II (Angelloz-Nicoud, P., and M. Binoux, supra, 1995; Figueroa, J. A., et al., supra, 1995) and IL-6 (Okamoto, M., et al., supra, 1997), other adipose cytokines, have been reported to stimulate androgen-independent and/or -dependent prostate cancer cell growth. Cell growth represents the balance between cell proliferation (cell cycle progression) and cell death (apoptosis). Since these cytokines have been shown anti-apoptotic, we expect that these adipose cytokines will stimulate cell survival (anti-apoptosis), as well as cell proliferation in prostate cancer cells. Moreover, our data show that prostate cancer cells express leptin, suggesting that leptin could stimulate prostate cancer cell growth in both paracrine and autocrine fashions as IGF-I (Pietrzkowski, Z., et al., supra, 1993), IGF-II (Angelloz-Nicoud, P., and M. Binoux, supra, 1995; Figueroa, J. A., et al., supra, 1995) and IL-6 (Okamoto, M., et al., supra, 1997) do. This led to a model: adipose cytokines, such as leptin, IGF-I, IGF-II and IL-6, interact with each other to stimulate both androgen-independent and androgen-dependent prostate cancer cell growth (cell cycle progression and survival) across paracrine and autocrine pathways via prostate cancer cell-adipocyte interaction. In addition, our recent research demonstrates that adiponectin inhibits prostate cancer cell growth.

Therefore, we propose the following clinical applications of our findings to manage prostate cancer targeting adipose cytokines and their signaling pathways. Clinically, our findings could be applied to therapeutic, preventive and diagnostic strategies.

B. Therapeutic Applications (Adipose Cytokine Ablation Therapy):

1. Androgen-Independent Prostate Cancer

Blockade of function of stimulator adipose cytokines such as IGF and IL6 leptin will suppress androgen-independent prostate cancer cell growth. Leptin functions can be blocked using several strategies, including small interfering RNAs (siRNAs) that inhibit expression of these cytokines or their cell surface receptors, neutralizing antibodies against these cytokines, antibodies against their receptors that block receptor function, and analogous peptides that inhibit binding of these cytokines to their receptors, and small molecules (chemicals) that bind the receptors and inhibit their functions.

By “leptin” we mean the molecule defined in Campfield, L. A., et al., Science 269:546-549, 1995 and Pelleymounter, M. A., et al., Science 269:540-543, 1995 and by “leptin function” we mean the metabolic effects of leptin administration.

In one embodiment of the invention, an siRNA inhibitor can be constructed and be delivered to target cells using siRNA oligonucleotides (Lewis, D. L., et al., Nat. Genet. 32:107-108, 2002; Song, E., et al., Nat. Med. 9:347-351, 2003, Sorensen, D. R., et al., J. Mol. Biol. 327:761-766, 2003), plasmids expressing siRNA (Brummelkamp, T. R., et al., Science 296:550-553, 2002; McCaffrey, A. P., et al., Nat. Biotechnol. 21:639-644, 2003; Miyagishi, M., and K. Taira, Nat. Biotechnol. 20:497-500, 2002; Yu, J. Y., et al., Proc. Natl. Acad. Sci. USA 99:6047-6052, 2002), or such viruses as adenovirus (Xia, H., et al., Nat. Biotechnol. 20:1006-1010, 2002), retrovirus (Barton, G. M., and R. Medzhitov, Proc. Natl. Acad. Sci. USA 99:14943-14945, 2002) and lentivirus (Tiscornia, G., et al., Proc. Natl. Acad. Sci. USA 100:1844-1848, 2003) expressing siRNA. siRNA can be administered to patients systemically (intravenously) or locally. Systemic administration of siRNA will target both prostate cancer cells and adipocytes. Local administration will target prostate cancer cells (tumors) specifically. For example, siRNA may be directly transferred to tumors using a gene gun (Sun, W. H., et al., Proc. Natl. Acad. Sci. USA 92:2889-2893, 1995). siRNA may be selectively delivered to tumors by injection to arteries that feed tumors. To treat liver metastasis, siRNA may be administered specifically to liver at high concentrations by isolated hepatic perfusion (IHP) (Alexander, H. R., Jr., et al., Cancer J. Sci. Am. 4:2-11, 1998). One would preferably create an siRNA according to the method reported by Elbashir, S., et al., Nature 411:494-498, 2001. One would typically generate 21-mer RNAs with 19 complementary nucleotides and 3′ terminal non-complementary dimmers of uridine. Target mRNA sequences need to start with two tandem adenines. Candidate cDNA sequences composed of the AA followed by 19 nucleotides will be compared to the GenBank human genome database to eliminate any sequences with significant homology to other genes. For example, Table 1 lists two sample siRNA molecules:

TABLE 1

1 START (56-58)
aggaaucgca gcgccaacgg uugcaaggcc caagaagccc auccugggaa ggaaaaugca
Target A (221-241)

(SEQ ID NO:1)

uuggggaacc ----------- cuccaaacag aaagucaccg guuuggacuu cauuccuggg
Target B (275-295)

(SEQ ID NO:2)

Cuccacccca uccugaccuu auccaagaug gaccagacac uggcagucua -----------

The two siRNAs above were designed against two different sequences of leptin mRNA within 250 bp downstream of the AUG start codon and were enzymatically synthesized using Silencer siRNA Construction Kit (Ambion). Both sequences are composed of the 5′ M and downstream 19 nucleotides, and demonstrate no significant honology to any other genes.

In another embodiment of the invention, antibodies against cytokines or their receptors can be administered intravenously. In case of liver metastasis, antibodies can also be administered exclusively to liver at high concentrations by IHP. Targets are stimulatory adipose cytokines, including leptin, IGF and IL-6 as well as their receptors. Anti-cytokine antibodies, which neutralize corresponding cytokines, are expected to reduce their blood concentrations systemically (i.v.) or locally (IHP). For intravenous administration of an anti-cytokine antibody, patients may be treated with 8 infusions of 8 mg/kg antibody biweekly during the initial dosing period, followed by adjustments in the dose and treatment interval for each patient (Nishimoto, N., et al., Blood, 2005). Anti-receptor antibodies, which block receptor functions, are expected to inhibit the stimulatory effects of cognate cytokines on cancer cells. For intravenous administration of an anti-receptor antibody, patients may be treated with a 4 mg/kg loading dose of the antibody followed by 2 mg/kg weekly (Bruno, R., et al., Cancer Chemother. Pharmacol., 2005).

Analogous peptides for leptin that inhibit binding of endogenous leptin to its receptors are expected to inhibit prostate cancer cell growth. These peptides can be administered systemically (intravenously) or locally. Peptides can be directly injected to tumors or selectively delivered to tumors by injection to arteries that feed tumors. Peptides can also be administered specifically to liver with metastatic tumors at high concentrations by IHP. We have designed and synthesized analogous peptides that are expected to inhibit binding of endogenous leptin to its receptor (Table 2 lists some examples). One of skill in the art could synthesize other analogues.

TABLE 2
Peptide Analogues of Leptin

| | | |
|---|---|---|
| L122-135 (WT): | EVVALSRLQGSLQD | (SEQ ID NO:3) |
| L122-135 (S12UD): | EVVALDRLQGSLQD | (SEQ ID NO:4) |
| L122-135 (R12IQ): | EVVALSQLQGSLQD | (SEQ ID NO:5) |
| L122-135 (S127D/ | EVVALDQLQGSLQD | (SEQ ID NO:6) |
| R128Q): | | |
| L33-44 (WT): | KQKVTGLDFIPG | (SEQ ID NO:7) |
| L33-44 (D40N): | KQKVTGLNFIPG | (SEQ ID NO:8) |

It has been shown that inhibition of autocrine IL-6 (Chung, T. D., et al., Prostate 38:199-207, 1999; Lou, W., et al., Prostate 42:239-42, 2000) or IGF (Pietrzkowski, Z., et al., supra, 1993) in prostate cancer cells suppresses androgen-independent prostate cancer cell growth. Therefore, inhibition of leptin, IL-6 and IGF in combination should be more effective in suppressing androgen-independent prostate cancer cell growth. Since these cytokines are all crucial in normal homeostasis, this combinatory “stimulatory adipose cytokine ablation therapy” may be applicable locally rather than systemically. Namely, tumors or tissues with metastatic tumors (i.e. liver) should be directly targeted.

We found JNK to mediate leptin-stimulated, androgen-independent prostate cancer cell proliferation. P13 kinase mediates leptin- and/or IGF-stumulated, androgen-independent prostate cancer cell proliferation via AKT activation. Signal transducer and activator of transcription 3 (STAT3) activation is required for androgen-independent prostate cancer cell growth (Mora, L. B., et al., Cancer Res. 62:6659-6666, 2002). Therefore, inhibition of JNK, P13 kinase or STAT3 alone or in combination in prostate cancer cells should be able to block androgen-independent prostate cancer cell growth efficiently. These molecules can be inhibited using pharmacological inhibitors (chemicals or peptides), dominant-negative mutants or siRNA. siRNA can be delivered to prostate cancer cells as described above. Dominant-negative mutants can be delivered using a plasmid expression vector or such viruses as adenovirus, retrovirus and lentivirus. Because these molecules play important roles in various physiological events, this strategy should selectively target tumors or tissues with metastatic tumors (i.e. injection into tumors by a gene gun, intraarterial injection to tumors, IHP) rather than systemically applied.

The use of antibodies, chemicals or gene manipulation is not the only way to carry out stimulatory adipose cytokine ablation. Circulating adipose cytokine levels can be decreased by controlling body fat through diet and exercise (see Preventive and Diagnostic Applications). Liposuction may be applicable to decrease body fat (stimulatory adipose cytokines) in a short term, too. Visceral fat tissue can be removed on prostate cancer surgery as well. Decrease in stimulatory adipose cytokine levels is expected to delay prostate cancer progression and sensitize cancer cells to conventional therapeutic strategies (see below).

The “stimulatory adipose cytokine ablation therapy” can be combined with other conventional strategies such as radiation therapy and chemotherapy. Inhibition of cytokines or their signaling pathways is expected to sensitize prostate cancer cells to radiation or conventional anti-cancer drugs. Thus, such combinatory therapies of conventional therapies and stimulatory adipose cytokine ablation should be much more effective than conventional therapies alone.

Our examples below indicate that adiponectin inhibits cell proliferation in androgen-independent prostate cancer cells. Therefore, another embodiment of the present invention is the use of increased amount of adiponectin or those of increased adiponectin function to treat prostate cancer cells.

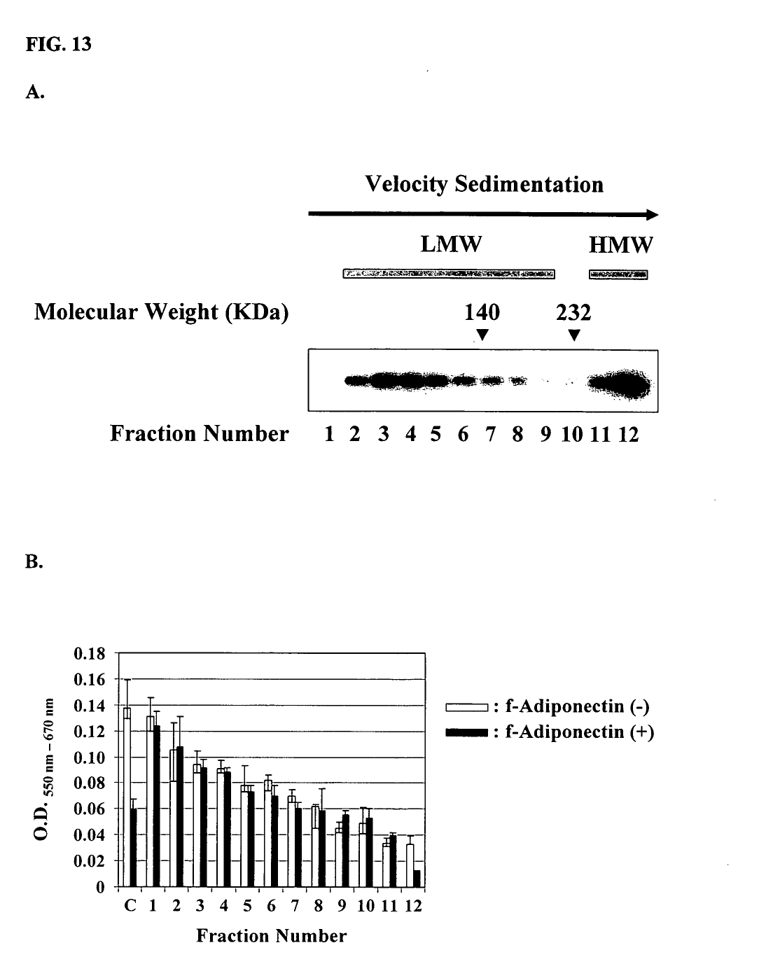

One would preferably increase adiponectin in the following manner: Adiponectin function can be increased by several strategies, including recominent adiponectin, adiponectin cDNA, and antibodies or small molecules (chemicals) that stimulate adiponectin receptor(s). For the use of recombinant adiponectin, the high molecule weight form of full-length adiponectin (f-adiponectin) can be purified (Kobayashi, H., et al., Cir. Res. 94(4):e21-31, 2004) and administered to patients since it is the form most inhibitory to prostate cancer cell growth (FIG. 13A). Adiponectin cDNA can be delivered to target cells using plasmids expressing cDNA (Heinzerling, L., et al., Hum. Gene Ther. 16(1):35-48, 2005) or such viruses as adenovirus (Cowen, D., et al., Clin. Cancer Res. 6(11):4402-4408, 2000), retrovirus (Hahn, W., et al., Gene Ther. 11 (9):739-745, 2004), and lentivirus (Kim, E. Y., et al., Biochem. Biophys. Res. Commun. 318(2):381-390, 2004) expressing cDNA. Recombinant adiponectin and adiponectin cDNA, as well as small molecules and antibodies that stimulate adiponectin receptor functions, can be administered to patients systemically (intravenously) or locally. Systemic application should be applicable to obese men since these agents, stimulating adiponectin functions, not only suppress prostate cancer but also ameliorate obesity and diabetes mellitus through acting on liver and muscle. Doses of recombinant adiponectin and adiponectin cDNA for systemic administration can be determined as circulating adiponectin levels are maintained at those for non-obese men (7.7±3.1 μg/ml) (Arita, Y., et al., Biochem. Biophys. Res. Commun. 257(1):79-83, 1999). Local administration will target prostate cancer cells (tumors) specifically. For example, plasmid DNA may be directly transferred to tumors using a gene gun (Sun, W. H., et al., Proc. Natl. Acad. Sci. USA 92:2889-2893, 1995). Recombinant adiponectin, plasmid DNA, chemicals and antibodies can be injected directly to tumors and/or their surrounding tissues. They may also be selectively delivered to tumors, including metastatic tumors, by injection to arteries that feed tumors. By “adiponectin” we mean the molecule disclosed in Pajvani, U. B., et al., J. Biol. Chem. 278:9073-9085, 2003; Kishida, K., et al., Biochem. Biophys. Res. Commun. 306:286-292, 2003. The high molecular weight form of f-adiponectin is the most effective form (FIG. 13A).

2. Androgen-Dependent Prostate Cancer

We found leptin to stimulate cell proliferation in androgen-dependent prostate cancer cells only in the presence of androgen but not in the absence of androgen. In other words, leptin sensitizes androgen-dependent cells to androgen. Interestingly, as with leptin, IGF-I has been reported to stimulate cell proliferation in androgen-dependent cells only in the presence of androgen (Iwamura, M., et al., Prostate 22:243-52, 1993). Therefore, the stimulatory adipose cytokine ablation therapy described above should be effective on androgen-dependent prostate cancer, as well as androgen-independent cancer, when combined with androgen ablation therapy. In addition, stimulation of adiponectin functions described above can be adjuvant therapy for androgen-dependent prostate cancer in combination with androgen ablation therapy.

C. Preventive and Diagnostic Applications:

Circulating levels of leptin (Chang, S., et al., Prostate 46:62-67, 2001; Stattin, P., et al., J. Clin. Endocrinol. Metab. 86:1341-1345, 2001), IL-6 (Drachenberg, D. E., et al., Prostate 41:127-133, 1999) or IGF-I (Chan, J. M., et al., Science 279:563-566, 1998; Chan, J. M., et al., J. Natl. Cancer Inst. 94:1099-1106, 2002) have been shown associated with prostate cancer. However, none of the methods is sensitive and specific enough to predict prostate cancer risk by itself. We expect that blood concentration profiles of multiple adipose cytokines will predict prostate cancer risk and prognosis more accurately. Furthermore, adipose cytokine profiling will be useful for prostate cancer diagnosis. The use of conventional diagnostic markers, such as prostate-specific antigen (PSA), in combination with adipose cytokine profiling may improve their reliability.

We propose to develop an adipose cytokine profiling kit. In one embodiment, this kit will profile blood levels of leptin, adiponectin, IGF-I, IGF-II, IL-6 and tumor necrosis factor-α (TNF-α), as well as PSA and DHT in combination (or subcombinations of these factors). TNF-α is another adipose cytokine and has been shown to play a role in the development of androgen-independency by prostate cancer cells (Mizokami, A., et al., J. Urol. 164:800-805, 2000). Plasma levels of these factors will be measured using mass spectrometry or such immunoreaction-based assays as enzyme-liked immunosorbent assay (ELISA), Immunofluorescence assay (IFA) and radioimmunoassay (RIA).

In summary, adipose cytokine profiling using this kit will be useful for the following:
i. Prevention of prostate cancer and prediction of prostate cancer risk
   We will prevent prostate cancer in men by controlling adiposity through diet and exercise with an adipose cytokine profile as a marker. Adipose cytokine profiles will predict prostate cancer risk.
ii. Diagnosis of prostate cancer
   Profiling adipose cytokine levels with the PSA level will improve reliability of PSA as a diagnostic marker.
iii. Prediction of prostate cancer prognosis
   Profiling adipose cytokine levels with PSA and DHT levels may predict prostate cancer prognosis well. Profiles of adipose cytokines, PSA and DHT may be useful to determine therapeutic strategies with prostate cancer patients.
iv. Prostate cancer therapy
   Suppressing stimulatory adipose cytokine levels with concomitant increase of adiponectin levels may delay prostate cancer progression (see Therapeutic Applications above). Adipose cytokine levels will be monitored using this kit.

In addition to circulating adipose cytokines, we will prepare visceral adipose tissue from patients due for prostate cancer surgery, and profile expression of these adipose cytokine genes in adipose tissue, as well as prostate cancer tissue, using real-time PCR analysis. Expression profiles of these cytokines by adipose and prostate cancer tissues may predict cancer prognosis well and be useful to determine therapeutic strategies with individual patients.

The circulating adipose cytokine profile of increase in stimulatory adpose cytokine levels with concomitant decrease of adiponectin levels is a diagnostic of prostate cancer and predicts cancer prognosis in combination with PSA and DHT levels. An expected profile would be: Leptin>12.4 ng/ml (Haffner, S. M., et al., Int. J. Obes. Relat. Metab. Disord. 20:904-908, 1996), IGF-1>270 NG/ML (Chan, J. M., et al., Science 279:563-566, 1998), IL-6>5.7 pg/ml (Drachenberg, D. E., et al., Prostate 41:127-133); and Adiponectin<5.3 μg/ml (Goktas, S., et al., Urology 65:1168-1172, 2005).

### EXAMPLES

A. Prostate Cancer Cell-Adipocyte Interaction: Leptin Mediates Androgen-Independent Prostate Cancer Cell Proliferation through c-Jun NH₂-Terminal Kinase

Summary

Prostate cancer is one of the leading causes of death among men in the United States, and acquisition of hormone resistance (androgen independence) by cancer cells is a fatal event during the natural history of prostate cancer. Obesity is another serious health problem and has been shown associated with prostate cancer. However, little is known about the molecular basis of this association. Here we show that factor(s) secreted from adipocytes stimulate prostate cancer cell proliferation. Leptin is one of the major adipose cytokines, and controls body weight homeostasis through food intake and energy expenditure. We identify leptin as a novel growth factor in androgen-independent prostate cancer cell growth. Strikingly, leptin stimulates cell proliferation specifically in androgen-independent DU145 and PC-3 prostate cancer cells but not in androgen-dependent LNCaP-FGC cells, although both cell types express functional leptin receptor isoforms. c-Jun NH₂-terminal kinase (JNK) has recently been shown to play a crucial role in obesity and insulin resistance. Intriguingly, leptin induces JNK activation in androgen-independent prostate cancer cells, and the pharmacological inhibition of JNK blocked the leptin stimulation of androgen-independent prostate cancer cell proliferation. This suggests that JNK activation is required for leptin-mediated, androgen-independent prostate cancer cell proliferation. Furthermore, other cytokines produced by adipocytes and critical for body weight homeostasis cooperate with leptin in androgen-independent prostate cancer cell proliferation: interleukin-6 and insulin-like growth factor I demonstrate additive and synergistic effects on the leptin stimulation of androgen-independent prostate cancer cell proliferation, respectively. Therefore, adipose cytokines, as well as JNK, are key mediators between obesity and hormone-resistant prostate cancer, and could be therapeutic targets.

Introduction

Prostate cancer is one of the leading causes of death among men in the United States. The disease is characterized by a prolonged natural history. Despite its relatively slow growth, a number of patients have persistent and/or recurrent disease. Initial treatment for many patients with recurrent disease is hormonal therapy to remove or decrease serum androgen as a potential growth stimulant for the prostate cancer. While this approach is initially effective in the majority of patients, ultimately the disease becomes resistant to the loss of hormones, returns and, in many cases, culminates in the death of the patient. Thus, it is desired to develop effective therapies and preventives for hormone-resistant (androgen-independent) prostate cancer.

Several lines of evidence indicate that obesity is a risk factor for prostate cancer. In particular, obesity is associated with clinical features characteristic of accelerated progression of prostate cancer: high mortality (Rodriguez, C., et al., supra, 2001), prostatectomy at a younger age with high grade and more pathologically advanced cancer (Amling, C. L., et al., supra, 2001) and tendency for progression of stage B1-D1 prostate cancer (Furuya, Y., et al., supra, 1998). Furthermore, abdominal adiposity, even in a lean population, has been shown associated with an increased risk of clinical prostate cancer (Hsing, A. W., et al., supra, 2000). However, little is known about the molecular basis of such associations. It is widely recognized that adipocytes produce various cytokines and hormones (Fruhbeck, G., et al., Am. J. Physiol. Endocrinol. Metab. 280:E827-E847, 2001). We hypothesize that such adipose factors may directly act on prostate cancer cells and accelerate cancer progression.

Leptin, one of the major cytokines produced by adipocytes, controls body weight homeostasis through food intake and energy expenditure (Campfield, L. A., et al., Science 269:546-596, 1995; Pelleymounter, M. A., et al., Science 269:540-543, 1995). Recessive mutations in the leptin (obese: ob) or its receptor (diabetes: db) gene result in profound obesity and type II diabetes mellitus (Zhang, Y., et al., Nature 372:425-432, 1994). In addition to body weight homeostasis, leptin is involved in various physiological events including reproduction (Zachow, R. J., and Magoffin, D. A. Endocrinology 138:847-850, 1997), hematopoiesis (Gainsford, T., et al., Proc. Natl. Acad. Sci USA 93:14564-14568, 1996), angiogenesis (Bouloumie, A., et al. Circ. Res. 83:1059-1066, 1998; Sierra-Honigmann, M. R., et al., Science 281:1683-1686, 1998), wound healing (Frank, S., et al., J. Clin. Invest. 106:501-509, 2000; Ring, B. D., et al., Endocrinology 141:446-449, 2000) and insulin secretion (Kieffer, T. J., et al., Diabetes 46:1087-1093, 1997; Shimizu, H., et al., Peptides 18:1263-1266, 1997), and has been demonstrated to regulate cell proliferation in various cells including breast cancer cells (Dieudonne, M. N., et al., Biochem. Biophys. Res. Commun. 293:622-628, 2002).

Upon binding to its cell surface receptor, leptin exerts cellular functions through the activation of downstream signaling pathways. The primary structure of leptin receptor revealed a single membrane spanning receptor of the class I cytokine receptor family (Tartaglia, L. A., et al., Cell 83:1263-1271, 1995). Four splice variants of leptin receptor have been identified in human: the full-length isoform huOB-R (Tartaglia, L. A., et al., supra, 1995) and the shorter isoforms huB219.1 to huB219.3 (Cioffi, J. A., et al., Nat. Med. 2:585-589, 1996) while five splice variants have been reported in normal mice: the full-length muOB-Rb and the shorter forms muOB-Ra, c, d and e (Friedman, J. M., and Halaas, J. L., Nature 395:763-770, 1998). The full-length isoform (muOB-Rb/huOB-R) can mediate the activation of signal transducer and activator of transcription (STAT) 3 (The abbreviations used are: STAT, signal transducer and activator of transcription; ERK, extracellular regulating kinase; JNK, c-Jun NH₂-terminal kinase; MAP, mitogen-activated protein; MTT, 3-(4,5-dimethylthiazol-2-yl)-2,5-diphenyl tetrazolium bromide; TNF-α, tumor necrosis factor-α; AP-1, activator protein-1; bZIP, basic region-leucine zipper; ATF, activating transcription factor; TRE, 12-O-tetradecanoylphorbol-13-acetate response element; CRE, cAMP response element; IL-6, interleukin-6; IGF, insulin-like growth factor; FBS, fetal bovine serum; GAPDH, glyceraldehyde-3-phosphate dehydrogenase) and extracellular regulating kinase (ERK) 1/2 pathways (Baumann, H., et al., Proc. Natl. Acad. Sci. USA 93:8374-8378, 1996; Bjorbaek, C., et al., J. Biol. Chem. 272:32686-32695, 1997; White, D. W., et al., J. Biol. Chem. 272:4065-4071, 1997), and the second longest isoform (muOB-Ra/huB219.3) is potent to activate ERK1/2 but not STAT3 (Bjorbaek, C., et al., supra, 1997; Bjorbaek, C., et al., J. Biol. Chem. 276:4747-4755, 2001). In addition, leptin has been shown capable of activating other pathways including the c-Jun NH₂-terminal kinase (JNK) pathway (Bouloumie, A., et al., Faseb J. 13:1231-1238, 1999). Expression of muOB-Rb/huOB-R has been demonstrated in limited tissues while shorter isoforms are expressed in most tissues (Cioffi, J. A., et al., supra, 1996); leptin receptor expression has been reported in normal (Cioffi, J. A., et al., supra, 1996) and malignant (Stattin, P., et al., supra, 2001) prostate epithelia, although expressed isoforms have not been identified.

JNK constitutes one of the four mammalian mitogen-activated protein (MAP) kinase families: ERK, JNK, p38 kinase and ERK5/big MAP kinase 1. JNK is activated in response to various stimuli and agents, including lipopolysaccharide endotoxin, inflammatory cytokines (e.g. interleukin-1 and tumor necrosis factor-α (TNF-α)), ionizing radiation, ultraviolet, hyperosmolarity, heat shock, and inhibitors of translation, such as anisomycin and cycloheximide (Derijard, B., et al., Cell 76:1025-1037, 1994; Hibi, M., et al., Genes Dev. 7:2135-2148, 1993; Kyriakis, J. M., et al., Nature 369:156-160, 1994; Raingeaud, J., et al., J. Biol. Chem. 270:7420-7426, 1995), translocates to the nucleus, and mediates the phosphorylation and activation of transcription factors, including c-Jun (Davis, R. J., Biochem. Soc. Symp. 64:1-12, 1999). Activated c-Jun participates in activator protein-1 (AP-1) formation. AP-1 is a group of dimeric basic region-leucine zipper (bZIP) proteins that pertain to the Jun (c-Jun, JunB, JunD), Fos (c-Fos, FosB, Fra-1, Fra-2), Maf (c-Maf, MafA, MafB, MafF/K/G, Nrl) and activating transcription factor (ATF) (ATF2, LRF1/ATF3, B-ATF, JDP1, JDP2) sub-families, which bind either the 12-O-tetradecanoylphorbol-13-acetate response element (TRE) or the cAMP response element (CRE) (Shaulian, E., and Karin, M., Nat. Cell. Biol. 4:E131-E136, 2002). JNK, as well as AP-1, contributes to the regulation of cell proliferation and apoptosis during various physiological and pathological events, including embryonic morphogenesis and tumor development (Davis, R. J., Cell 103:239-252, 2000). Intriguingly, JNK has recently been demonstrated to play a crucial role in obesity and insulin resistance (Hirosumi, J., et al., Nature 420:333-336, 2002). JNK2 has been shown involved in cell proliferation (cell cycle progression) and survival in androgen-independent prostate cancer PC-3 cells (Potapova, O., et al., Cancer Res. 62:3257-3263, 2002). However, to our best knowledge, no cytokines or growth factors have been reported to trigger the cell cycle progression or cell survival of prostate cancer cells via JNK activation.

Leptin is not the only cytokine that is produced by adipocytes and plays a role both in body weight homeostasis and androgen-independent prostate cancer cell growth. Interleukin-6 (IL-6) is secreted from adipocytes in addition to other cell types (Fruhbeck, G., et al., supra, 2001). As with leptin, serum IL-6 levels correlate to body mass index (Fried, S. K., et al., J. Clin. Endocrinol. Metab. 83:847-850, 1998; Mohamed-Ali, V., et al., J. Clin. Endocrinol. Metab. 82:4196-4200, 1997), and IL-6 knockout mice develop mature-onset obesity with an increase in subcutaneous fat mass (Wallenius, V., et al., Nat. Med. 8:75-79, 2002). Interestingly, serum IL-6 levels are remarkably elevated in patients with clinically evident hormone-resistant prostate cancer as compared to those with hormone-dependent cancer (Drachenberg, D. E., et al., supra, 1999). Consistent with the clinical result, IL-6 is secreted by androgen-independent prostate cancer cells but not by androgen-dependent LNCaP cells (Chung, T. D., et al., supra, 1999; Okamoto, M., et al., supra, 1997). This autocrine IL-6 (Chung, T. D., et al., supra, 1999; Okamoto, M., et al., supra, 1997), as well as exogenous IL-6 (Okamoto, M., et al., supra, 1997), stimulates androgen-independent prostate cancer cell growth. This suggests that IL-6 can stimulate androgen-independent prostate cancer cell growth in both autocrine and paracrine manners. Adipocytes secrete insulin-like growth factor (IGF)-I, too (Fruhbeck, G., et al., supra, 2001; Longo, K. A., et al., J. Biol. Chem. 277:38239-38244, 2002). IGF-I is implicated in energy homeostasis (Platz, E. A., J. Nutr. 132(11 Suppl):3471S-3481S, 2002). Plasma IGF-I levels are associated with prostate cancer risk (Chan, J. M., et al., supra, 1998) and may predict the risk of developing advanced-stage prostate cancer (Chan, J. M., et al., supra, 2002). Like IL-6, IGF-I is secreted by prostate cancer cells and can promote androgen-independent prostate cancer cell growth in both autocrine and paracrine fashions (Kaicer, E. K., et al., Growth Factors 4:231-237, 1991; Pietrzkowski, Z., et al., supra, 1993).

In this study, we employ DU145 and PC-3 cells as models for androgen-independent prostate cancer cells, and LNCaP-FGC cells for androgen-dependent prostate cancer cells, and demonstrate that adipose factors stimulate prostate cancer cell proliferation. We identify leptin as a novel growth factor in androgen-independent prostate cancer cell proliferation, and show that JNK mediates leptin-stimulated, androgen-independent prostate cancer cell proliferation. Furthermore, we identify collaboration of leptin with IL-6 and IGF-I in androgen-independent prostate cancer cell proliferation. Thus, adipose cytokines, such as leptin, IL-6 and IGF-I, are key mediators between prostate cancer and obesity.

Materials and Methods

Cytokines, JNK Inhibitor and Antibodies

Recombinant human IL-6 and leptin were purchased from R&D Systems Inc. (Minneapolis, Minn., USA). SP600125, a pharmacological JNK inhibitor, and anisomycin were purchased from Tocris Cookson, Inc. (Ellisville, Mo., USA). The SAPK/JNK Assay Kit, and anti-JNK, anti-c-Jun, anti-phospho-c-Jun (Ser-63) and anti-phospho-c-Jun (Ser-73) polyclonal antibodies were purchased from Cell Signaling Technology, Inc. (Beverly, Mass., USA).

Prostate Cancer Cells and Culture Conditions

Human prostate cell lines DU145, PC-3 and LNCaP-FGC were purchased from the American Type Culture Collection (Manassas, Va., USA). DU145 cells were cultured in DMEM supplemented with 10% FBS plus penicillin (100 units/ml) and streptomycin (100 μg/ml). PC-3 and LNCaP-FGC cells were cultured in RPMI 1640 supplemented with 10% fetal bovine serum (FBS) and antibiotics.

In Vitro Adipocyte Differentiation

Human primary preadipocytes were purchased from Bio Whittaker Inc. (Walkersville, Md., USA) and cultured in preadipocyte basal growth medium (PBM) (Bio Whittaker Inc.) supplemented with 10% FBS, 2 mM L-glutamine and antibiotics. To induce the differentiation of preadipocytes into mature adipocytes, confluent preadipocytes plates (3.3×10⁴/cm²) were cultured in PBM plus 10% FBS and antibiotics for 24 hours and then switched to PBM plus 10% FBS, antibiotics, 10 μg/ml insulin, 500 μM isobutyl-methylxanthine, 1 μM dexamethasone and 200 μM indomethacin. After 9 days of incubation, the cells were washed three times with phosphate-buffered saline (PBS) and then incubated with PBM without serum for 24 hours; the conditioned medium was collected, centrifuged at 1,000×g to remove cell debris and stored at ˜0° C. until use. To identify fat accumulation, cells were fixed with 4% paraformaldehyde, stained with Oil-Red-O (Sigma Chemical Co., St. Louise, Mo., USA) and observed under a bright-field microscope as described previously (Calkhoven, C. F., et al., Genes Dev. 14:1920-1932, 2000).

Thymidine Incorporation Assay

DU145 (1.5×10⁵/well), PC-3 (1.0×10⁵/well) and LNCaP-FGC (2.0×10⁵/well) cells were seeded into 24-well plates 24 hours before serum-starvation. Cells were deprived of serum for 48 hours, followed by incubation with leptin at indicated concentrations or 100 ng/ml IL-6 for 20 hours. In some experiments, cells were pretreated with SP600125 prior to cytokine stimulation as described below. Cells in each well were pulsed with 1 μCi of [³H] thymidine (Amersham Pharmacia Biotech Inc., Piscataway, N.J., USA) during the final 5 hours, washed once with PBS, fixed with cold 5% trichloroacetic acid, solubilized with 0.25 M NaOH, and counted in scintillant. Each experiment was performed in quadruplicate, and the values are reported as mean±SD.

MTT Assay

DU145 (1×10³/well), PC-3 (1×10³/well) and LNCaP-FGC (1.5×10³/well) cells were seeded in 96-well plates 24 hours prior to serum starvation. Cells were starved of serum for 24 hours and then incubated in the serum-free medium with or without 12.5 μg/ml leptin for 5 days. 3-(4,5-dimethylthiazol-2-yl)-2,5-diphenyl tetrazolium bromide (MTT) was added to each well to a concentration of 0.5 mg/ml. After 3-hour incubation at 37° C., cells were lysed in 50% dimethylformamide and 20% SDS at 37° C. Optical densities (O.D.) at 550 nm and 670 nm were measured by a plate reader, and differential O.D. between 550 nm and 670 nm (O.D. 550 nm minus O.D. 670 nm) were determined. Each experiment was performed in quadruplicate, and the values are reported as mean±SD.

Quantitative RT-PCR Analysis

Total RNA was isolated by the TRIZOL standard technique. To evaluate mRNA expression patterns of leptin receptor isoforms (huOB-R, huB219.1-3), total RNA was extracted from DU145, PC-3 and LNCaP-FGC cells incubated without serum for 24 hours. Two micrograms of each RNA were used to generate cDNA in a 20-μl-reaction mixture, according to the Omuniscript Reverse Transcriptase protocol (QIAGEN Inc., Valencia, Calif., USA). The reaction mixture for PCR (20 μl) contained 1×PCR buffer (Roche Molecular Biochemicals, Mannheim, Germany), 0.1-0.5 μM of each primer, 0.2 μM of each dNTP (QIAGEN Inc., Valencia, Calif., USA), 1.0 U of Taq DNA polymerase (Roche Molecular Biochemicals) and 1 μl of the cDNA solution. PCR was performed with an initial denaturing at 94° C. for 2 minutes, followed by 19-39 cycles consisting of denaturing at 94° C. for 30 seconds, annealing at 55-62° C. for 30 seconds, and extending at 72° C. for 1 minute. Amounts of PCR products were compared after cycles of reactions that exhibited DNA amplification in a linear range. The same forward primer (5′-TTG TGC CAG TAA TTA TTT CCT CTT-3′) was used for amplification of all the leptin receptors with different reverse primers: huB219.1 (5′-CTG TGG CCT TCC GCA GTG-3′), huB219.2 (5′-ACC TCC ACC CAG TAG TTC CTT-3′), huB219.3 (5′-AGT TGG CAC ATT GGG TTC AT-3′) and huOB-R (5′-CTG ATC AGC GTG GCG TAT TT-3′) (Mix, H., et al., Gut 47:481-486, 2000). The PCR reactions were normalized using glyceraldehyde-3-phosphate dehydrogenase (GAPDH) as an internal standard. Forward and reverse primers used for GAPDH were 5′-TGA AGG TCG GTG TCA ACG GAT TTG GC-3′ and 5′-CAT GTA GGC CAT GAG GTC CAC CAC-3′, respectively. PCR products were analyzed by electrophoresis on a 2.0% gel with ethidium bromide staining.

Cell Lysate Preparation

Eighty-percent confluent prostate cancer cells were starved in the serum-free medium for 24 hours and then treated by 12.5 μg/ml leptin or 10 μg/ml anisomycin for the indicated periods. In some experiments, cells were pretreated with SP600125 prior to cytokine stimulation as described below. Cells were washed twice with cold PBS, harvested by a scraper and centrifuged for 10 minutes at 700×g at 4° C. The pellet was homogenized in the cell lysis buffer (20 mM HEPES, pH 7.9, 300 mM NaCl, 1 mM EDTA, 15% glycerol, 0.5% NP-40, 1 mM Na₃VO₄, 1 μg/ml leupeptin, 5 μg/ml aprotinin, 1 μg/ml pepstatin and 1 mM PMSF) and incubated at 4° C. with gentle rocking for 30 minutes. This homogenate was centrifuged for 5 minutes at 15,000×g, and the supernatant was saved as a cell lysate and stored at −80° C. until use.

Western Blot Analysis of c-Jun Phosphorylation

Cell lysates containing 100 μg of protein were electrophoresed on 10% SDS-polyacrylamide gels and electrotransferred to PVDF membranes. Following the supplier's instructions, the membranes were washed, blocked and hybridized with anti-phospho-c-Jun (Ser-63 or Ser-73) polyclonal antibody that specifically recognizes a phosphorylated form of c-Jun. Bands were visualized using a Lumiglo reagent (Cell Signaling Technology Inc.). The membranes were then stripped and re-hybridized with anti-c-Jun antibody that detects both phosphorylated and unphosphorylated forms to normalize sample variations.

In Vitro JNK Assay

JNK activity was measured using the SAPK/JNK Assay Kit (Cell Signaling Technology Inc.) according to the manufacture's instructions. Briefly, JNK was precipitated in 250 μl of cell lysates (250 μg protein) with 2 μg of glutathione Sepharose beads immobilized by a recombinant fusion protein of the c-Jun N-terminus (1-89) and glutathione S-transferase as a JNK substrate. After being washed with the cell lysis buffer 3 times, the precipitates were incubated in 50 μl of the kinase buffer containing 100 μM ATP at 30° C. for 30 minutes. The reaction was stopped by addition of the SDS sample buffer. The N-terminal c-Jun (1-89) fusion protein was separated by SDS-polyacrylamide gel electrophoresis, and its phoshporylation was determined by Western blot analysis using anti-phospho-c-Jun (Ser-63) polyclonal antibody as described above. JNK protein levels in cell lysates were also measured by Western blot analysis using anti-JNK antibody to normalize sample variations.

Pharmacological Inhibition of JNK in Prostate Cancer Cells

SP600125 was dissolved in DMSO to make a stock solution. Eighty-percent confluent prostate cancer cells were serum-starved for 24 hours and pretreated with SP600125 at indicated concentrations for 30 minutes, followed by leptin treatment (12.5 μg/ml): 15 minutes for Western blot analysis or 20 hours for thymidine incorporation assays. DMSO-treated cells were employed as controls.

Results

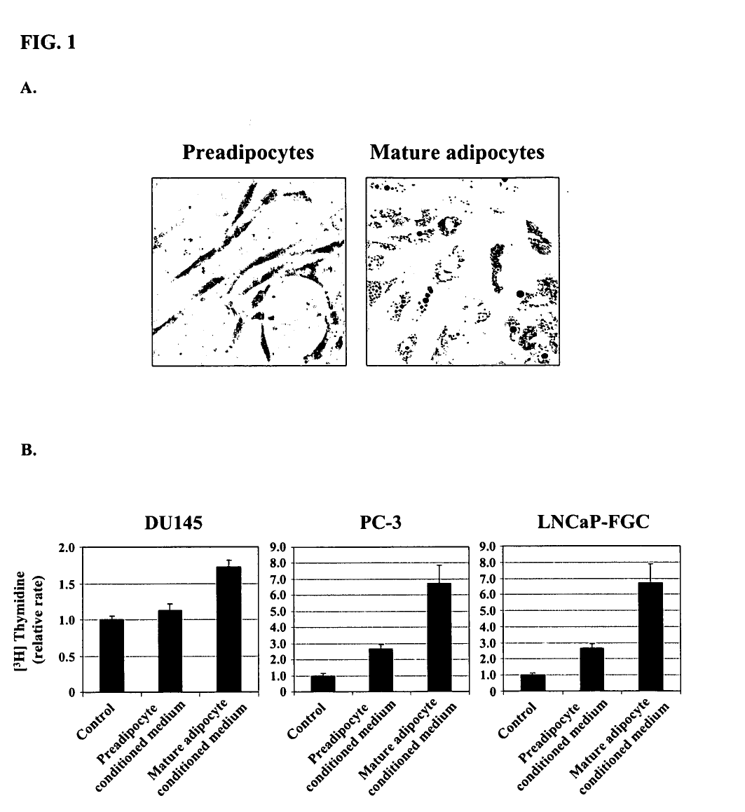

To address our hypothesis that factor(s) produced by adipocytes may accelerate prostate cancer progression, we examined whether conditioned medium from human primary adipocyte culture stimulates cell proliferation in prostate cancer cells using a thymidine incorporation assay. We differentiated primary human preadipocytes to mature adipocytes in vitro by treating cells with insulin, isobutyl-methylxanthine, dexamethasone, and indomethacin as described above. Fat staining with Oil-Red-O demonstrates that cells accumulated fat droplets only after the treatment (FIG. 1A), indicating they successfully differentiated from preadipocytes to mature adipocytes. Prostate cancer cells were treated with conditioned media from these two cell types. Strikingly, the conditioned medium from mature adipocyte culture stimulated thymidine incorporation remarkably in both androgen-independent DU145 cells and androgen-dependent LNCaP-FGC cells, while that from preadipocyte culture exhibited only modest effects (FIG. 1B). This suggests strongly that adipose factor(s) promote cell proliferation both in androgen-independent and -dependent prostate cancer cells.

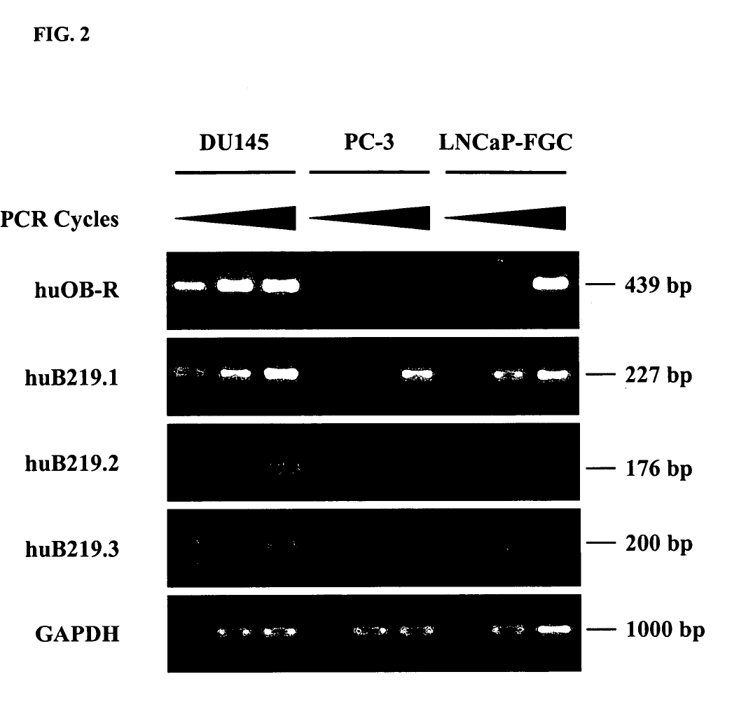

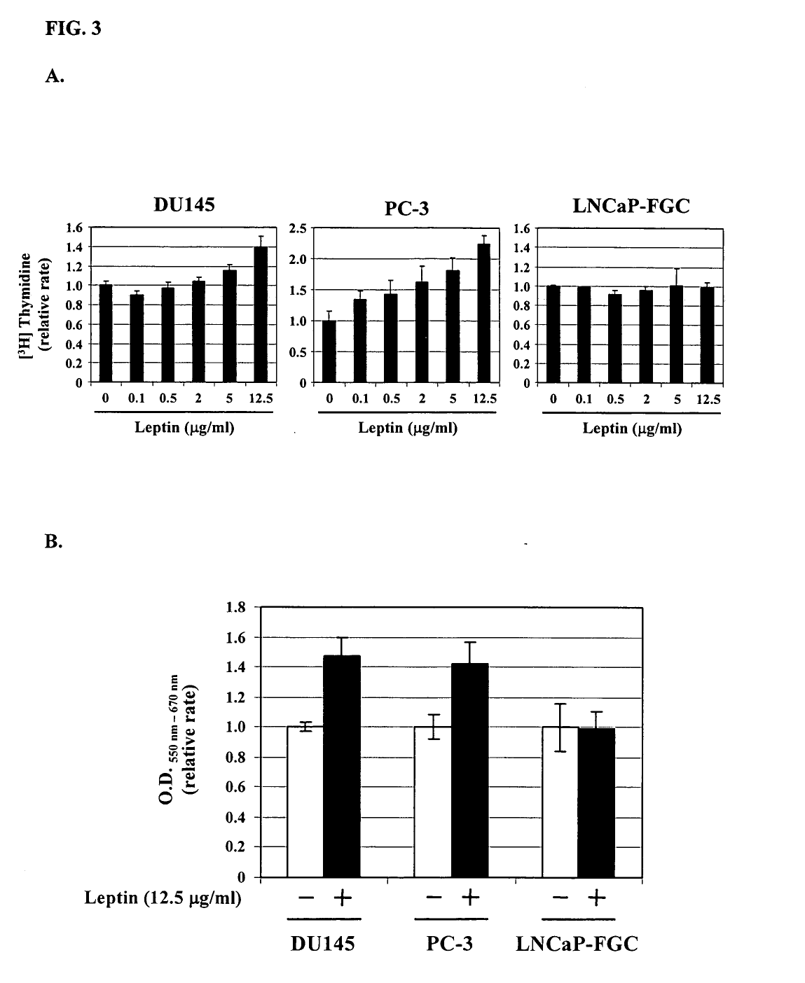

Leptin is one of the major cytokines secreted from adipocytes (Campfield, L. A., et al., supra, 1995; Pelleymounter, M. A., et al., supra, 1995). We profiled the expression of leptin receptor isoforms in the two prostate cancer cell lines. FIG. 2 demonstrates that DU145, PC-3 and LNCaP-FGC cells all express both of the two functional isoforms (huOB-R and huB219.3) (Cioffi, J. A., et al., supra, 1996). This is consistent with a previous report that human prostate cancer tissue expresses leptin receptor (Stattin, P., et al., supra, 2001). To determine whether leptin regulates prostate cancer cell proliferation, we tested the effects of added leptin on thymidine incorporation in these prostate cancer cells. Leptin stimulated thymidine incorporation remarkably in a dose-dependent fashion at concentrations up to 12.5 μg/ml in androgen-independent DU145 and PC-3 cells (FIG. 3A). Leptin might stimulate thymidine incorporation with no effect on actual cell proliferation in prostate cancer cells. To exclude this possibility, we performed the MTT assay, a colorimetric cell growth assay that measures the reduction of MTT by mitochondrial succinate dehydrogenase and, thus, detects living cells. Consistent with thymidine incorporation, 12.5 μg/ml leptin increased living cells in an androgen-independent manner in DU145 and PC-3 cells (FIG. 3B). This confirms that leptin-stimulated thymidine incorporation correlates with leptin-stimulated cell growth in androgen-independent prostate cancer cells. This is the first evidence that leptin functions as a growth factor in androgen-independent prostate cancer cell growth. In contrast, leptin did not influence either thymidine incorporation or live cell mass in androgen-dependent LNCaP-FGC cells (FIGS. 3A and B).

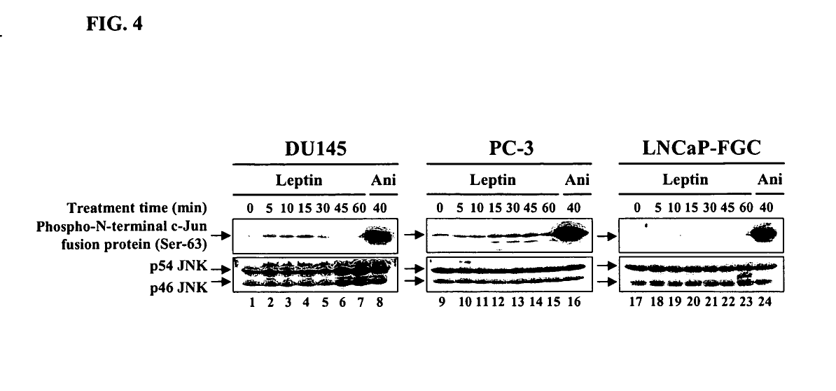

Leptin is potent to activate various intracellular signaling pathways. We examined activation of these pathways in prostate cancer cells after being treated with leptin for various periods up to 60 minutes. FIG. 4 shows that 12.5 μg/ml leptin stimulated JNK activity in DU145 and PC-3 cells. JNK activity was constitutively detected at low levels in these two cell lines. Added leptin induced JNK activation in DU145 cells, peaking 15 minutes after addition of leptin and returning to the basal level 45 minutes after. JNK activation by leptin also peaked after a 15-minute incubation in PC-3 cells. Constitutive JNK activation was detectable in LNCaP-FGC cells as well. However, leptin did not influence JNK activity in LNCaP-FGC cells. Therefore, JNK activation correlates to androgen-independent prostate cancer cell proliferation in response to leptin.

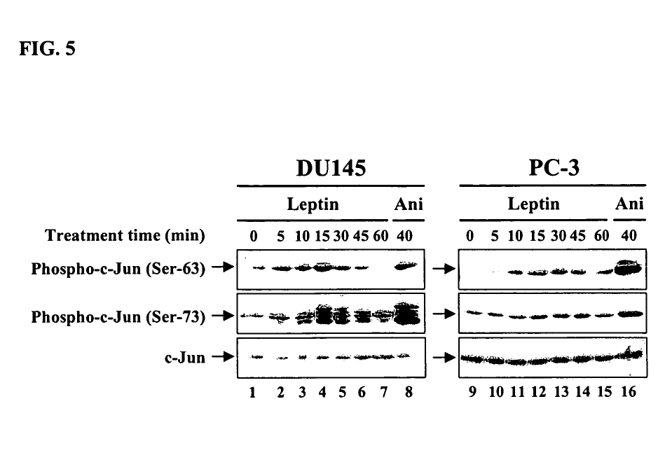

c-Jun is an endogenous substrate for JNK. JNK phosphorylates c-Jun at Ser-63 and Ser-73 in its transactivation domain (Shaulian, E., and Karin, M., supra, 2002), and phosphorylation of these Ser residues is crucial for the transactivation and DNA binding activity of c-Jun (Shaulian, E., and Karin, M., supra, 2002). We examined c-Jun phosphorylation in DU145 and PC-3 cells during leptin-stimulated androgen-independent cell proliferation. c-Jun was constitutively phosphorylated on both Ser residues at low levels in DU145 and PC-3 cells, and addition of leptin (12.5 μg/ml) stimulated c-Jun phosphorylation on these Ser residues in correlation to JNK activation (FIG. 5).

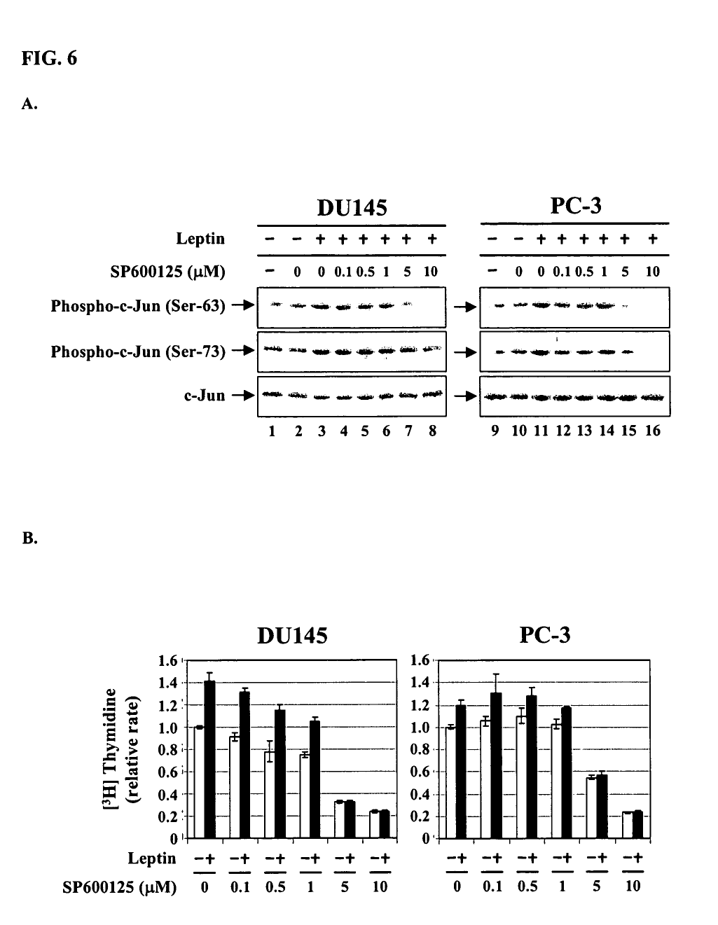

To determine whether JNK activation mediates leptin-stimulated androgen-independent cell proliferation, we analyzed the effects of pharmacological JNK inhibition on the leptin stimulation of thymidine incorporation, as well as c-Jun phosphorylation, in DU145 and PC-3 cells. FIG. 6A shows that at concentrations from 0 to 10 μM, SP600125, a pharmacological JNK inhibitor (Bennett, B. L., et al., Proc. Natl. Acad. Sci. USA 98:13681-13686, 2001; Han, Z., et al., J. Clin. Invest. 108:73-81, 2001; Schnabl, B., et al., Hepatology 34:953-963, 2001), suppressed the leptin stimulation of c-Jun phosphorylation on Ser-63 and Ser-73 in a dose-dependent manner in both DU145 and PC-3 cells. Likewise, SP600125 inhibited leptin-stimulated cell proliferation at the same range of concentrations in these two cell lines (FIG. 6B). These indicate that leptin mediates androgen-independent cell proliferation in DU145 and PC-3 cells through JNK activation, most likely via c-Jun phosphorylation. It is interesting to note that Ser-63 phosphorylation is more sensitive to JNK inhibition and correlates better to the SP600125 inhibition of leptin-stimulated cell proliferation as compared to Ser-73 phosphorylation. This may suggest differential roles of the two phosphorylated Ser residues on c-Jun in cell growth regulation and is being studied further in our laboratory. In addition, SP600125 inhibited the basal levels of cell proliferation in DU145 and PC-3 cells (FIG. 6B). This is consistent with a recent report showing that JNK2 gene inhibition by antisense oligonuleotides remarkably suppressed cell proliferation and survival in PC-3 cells (Potapova, O., et al., supra, 2002). To the best of our knowledge, leptin is the first cytokine that has been demonstrated to stimulate prostate cancer cell growth via JNK activation.

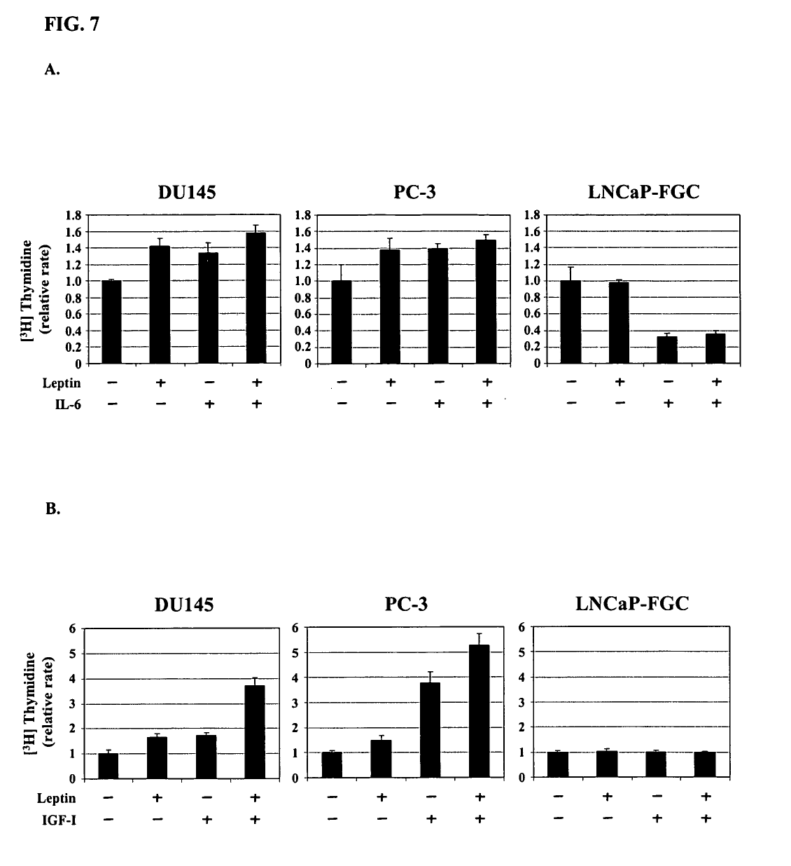

To determine the interaction of leptin with other adipose cytokines in androgen-independent prostate cancer cell proliferation, we treated cells with leptin together with IL-6 or IGF-I. As previously shown (Okamoto, M., et al., supra, 1997; Mori, S., et al., Biochem. Biophys. Res. Commun. 257:609-614, 1999), IL-6 alone stimulated cell proliferation in androgen-independent DU145 and PC-3 cells but inhibited in androgen-dependent LNCaP-FGC cells (FIG. 7A). Leptin showed additive effects on the IL-6 stimulation of androgen-independent prostate cancer cell proliferation in DU145 and PC-3 cells, but it did not influence the IL-6-induced inhibition of LNCaP-FGC cell proliferation (FIG. 7A). Consistent with previous reports (Iwamura, M., et al., supra, 1993), IGF-I alone augmented cell proliferation in DU145 and PC-3 cells but did not affect LNCaP-FGC cell proliferation (FIG. 7B). Strikingly, leptin exhibited drastic, synergistic effects on the IGF-I stimulation of prostate cancer cell proliferation in DU145 and PC-3 cells, while co-treatment with leptin and IGF-I did not influence LNCaP-FGC cell proliferation (FIG. 7B). These findings strongly indicate that leptin plays a crucial role in androgen-independent prostate cancer cell growth in collaboration with other adipose cytokines.

Discussion

Conditioned medium from adipocyte culture, which should contain various adipose factors, stimulated cell proliferation in prostate cancer cells (FIG. 1B). In combination with clinical studies demonstrating the association of obesity with prostate cancer (Rodriguez, C., et al., supra, 2001; Amling, C. L., et al., supra, 2001; Furuya, Y., et al., supra, 1998), this strongly suggests that obesity may promote prostate cancer cell growth mediated by adipose factors. In the present study, we demonstrated that leptin, one of the major adipose cytokines, which controls body weight homeostasis through food intake and energy expenditure (Campfield, L. A., et al., supra, 1995; Pelleymounter, M. A., et al., supra, 1995), stimulated androgen-independent prostate cancer cell proliferation (FIGS. 3A and B).

In addition to leptin, such adipose cytokines as IL-6 and IGF-I have been shown associated with both body weight homeostasis (Fried, S. K., et al., supra, 1998; Mohamed-Ali, V., et al., supra, 1997; Wallenius, V., et al., supra, 2002; Platz, E. A., supra, 2002) and prostate cancer progression (Drachenberg, D. E., et al., supra, 1999; Chan, J. M., et al., supra, 1998; Chan, J. M., et al., supra, 2002) as described above. Interestingly, as with leptin, IL-6 (Chung, T. D., et al., supra, 1999; Okamoto, M., et al., supra, 1997; Mori, S., et al., supra, 1999) or IGF-I (Iwamura, M., et al., supra, 1993) alone augments prostate cancer cell growth in androgen-independent DU145 and PC-3 cells but not in androgen-dependent LNCaP-FGC cells (FIGS. 7A and 7B). Furthermore, we identified collaboration of IL-6 and IGF-I with leptin in the androgen-independent stimulation of prostate cancer cell proliferation. IL-6 showed additive effects on the leptin stimulation of androgen-independent prostate cancer cell proliferation (FIG. 7A) while IGF-I exhibited drastic, synergistic effects (FIG. 7B). This suggests that adipose cytokines may collaborate with each other to promote hormone-resistant tumor growth in prostate cancer patients either in an additive or synergistic manner. Circulating adipose cytokines may affect prostate cancer cell growth to some degree. However, paracrine adipose cytokines may be more important when cancer cells are exposed to adipose tissue, providing cancer cells with higher concentrations of cytokines. Prostate cancer cells would be exposed to adipose tissue when they invade the retropubic fat pad or metastasize to bone marrow or lymph nodes. This is consistent with the evidence, cited above, that obesity seems associated with the accelerated progression of prostate cancer (Rodriguez, C., et al., supra, 2001; Amling, C. L., et al., supra, 2001; Furuya, Y., et al., supra, 1998).

Leptin, IL-6 or IGF-I alone showed no stimulatory effects on cell proliferation in androgen-dependent LNCaP-FGC cells (FIGS. 3A, 3B, 7A and 7B) although the conditioned medium from adipocyte culture stimulated cell proliferation in both LNCaP-FGC cells and androgen-independent DU145 and PC-3 cells (FIG. 1B). This suggests that adipose cytokines other than leptin, IL-6 and IGF-I contribute to growth stimulation in LNCaP-FGC cells.

Adipose cytokines may contribute not only to androgen-independent cell growth but also to the development of androgen-independency by prostate cancer cells. TNF-α is another cytokine that is produced by adipocytes (Fruhbeck, G., et al., supra, 2001; Hotamisligil, G. S., et al., Science 259:87-91, 1993) and involved in the development of obesity-linked insulin resistance (Hotamisligil, G. S., et al., supra, 1993). It has been reported that TNF-α repressed androgen-sensitivity in androgen-dependent LNCaP cells through down-regulation of androgen receptor expression (Mizokami, A., et al., J. Urol. 164:800-805, 2000), suggesting that TNF-α may play a role in the initiation of an androgen-independent state in prostate cancer cells.

In addition to adipose cytokines such as leptin, IL-6 and IGF-I, JNK has recently been demonstrated to play a central role in obesity and insulin resistance. JNK activity is significantly elevated in obesity, and JNK1 gene deletion results in decreased adiposity, remarkably improved insulin sensitivity and enhanced insulin receptor signaling capacity in mice (Hirosumi, J., et al., supra, 2002). It has been reported that JNK2 is crucial in prostate cancer cell proliferation and survival (Potapova, O., et al., supra, 2002). Here we identify leptin as an upstream activator of JNK in androgen-independent prostate cancer DU145 and PC-3 cells (FIG. 4) and show that leptin promotes androgen-independent cell proliferation via JNK activation (FIG. 6B). Therefore, leptin and JNK, as well as IL-6 and IGF-I, are common key players in obesity/insulin resistance and hormone resistant prostate cancer. These molecules may link prostate cancer, particularly in the hormone resistant stage, and obesity/insulin resistance in etiology. Thus, they could be common therapeutic targets, as well as prognostic markers, for these diseases. Since such dietary factors as animal fat consumption, which is apparently associated with obesity, are linked to clinical prostate cancer (Key, T., Cancer Surv. 23:6, 1995), we may be able to prevent and treat prostate cancer, especially in the hormone-resistant stage, by controlling adiposity through diet with these molecules as markers.

B. Adiponectin Activates c-Jun NH₂-Terminal Kinase and Inhibits Signal Transducer and Activator of Transcription 3

Abstract

Adiponectin, a major adipose cytokine, plays a crucial role in inhibition of metabolic syndrome through acting on such cell types as muscle cells and hepatocytes. Furthermore, evidence suggests that adiponectin may influence cancer pathogenesis. Adiponectin occurs in non-proteolytic (full-length adiponectin: f-adiponectin) and proteolytic (globular adiponectin: g-adiponectin) forms in various oligomeric states. Different forms of adiponectin show distinct biological effects through differential activation of downstream signaling pathways. Here we identify c-Jun NH₂-terminal kinase (JNK) and signal transducer and activator of transcription 3 (STAT3) as common downstream effectors of f- and g-adiponectin. f- and g-Adiponectin both stimulate JNK activation in prostate cancer DU145, PC-3 and LNCaP-FGC cells, hepatocellular carcinoma HepG2 cells, and C2C12 myoblasts. Furthermore, both f- and g-adiponectin drastically suppress constitutive STAT3 activation in DU145 and HepG2 cells. These suggest that JNK and STAT3 may constitute a universal signaling pathway to mediate adiponectin's pathophysiological effects on metabolic syndrome and cancer.

Introduction

Adiponectin is a cytokine encoded by a gene that is expressed most abundantly and highly specifically in adipose tissue (K. Maeda, et al., Biochem. Biophys. Res. Commun. 221:286-289, 1996; E. Hu, et al., J. Biol. Chem. 271:10697-10703, 1996; P. E. Scherer, et al., J. Biol. Chem. 270:26746-26749, 1995). Plasma levels of adiponectin correlate negatively with body mass index (Y. Arita, et al., Biochem. Biophys. Res. Commun. 257:79-83, 1999) and visceral fat accumulation (M. Cnop, et al., Diabetologia 46:459-469, 2003). Furthermore, low levels of plasma adiponectin are associated with such obesity-related disorders as diabetes mellitus (K. Hotta, et al., Arterioscler. Thromb. Vasc. Biol. 120:595-1599, 2000), coronary artery (atherosclerotic) disease (M. Kumada, et al., Arterioscler. Thromb. Vasc. Biol. 23:85-89, 2003) and hypertension [Y. Iwashima, et al., Hypertension 43:1318-1323, 2004). Mouse model studies have demonstrated crucial roles of adiponectin in pathogenic alterations of these disorders (J. Fruebis, et al., Proc. Natl. Acad. Sci. USA 98:2005-2010, 2001; A. H. Berg, et al., Nat. Med. 7:947-953, 2001; N. Maeda, et al., supra, 2002; N. Ouchi, et al., Hypertension 42:231-234, 2003). In addition, several lines of evidence indicate that adiponectin may influence cancer pathogenesis. Circulating adiponectin levels are inversely associated with an increased risk of breast (C. Mantzoros, et al., J. Clin. Endocrinol. Metab. 89:1102-1107, 2004; Y. Miyoshi, et al., Clin. Cancer Res. 9:5699-5704, 2003) and endometrial (E. Petridou, et al., J. Clin. Endocrinol. Metab. 88:993-997, 2003; L. Dal Maso, et al., J. Clin. Endocrinol. Metab. 89:1160-1163, 2004) cancer, and breast tumors arising in women with low adiponectin levels are more likely to show a biologically aggressive phenotype (Y. Miyoshi, et al., Clin. Cancer Res. 9:5699-5704, 2003). Furthermore, adiponectin has been shown to inhibit cell proliferation and induce apoptosis in leukemia cells (T. Yokota, et al., Blood 96:1723-1732, 2000) and to suppress tumor growth in mice, most likely due to inhibition of neovascularization through suppression of endothelial cell proliferation, migration and survival (E. Brakenhielm, et al., Proc. Natl. Acad. Sci. USA 101:2476-2481, 2004).

Adiponectin is found in both non-proteolytic (full-length adiponectin: f-adiponectin) and proteolytic forms. f-Adiponectin, a 30-kDa polypeptide, is comprised of an amino-terminal signal sequence, a variable domain, a collagen-like domain and a carboxyl-terminal globular domain (K. Maeda, et al., Biochem. Biophys. Res. Commun. 221:286-289, 1996; E. Hu, et al., J. Biol. Chem. 270:26746-26749, 1995; Y. Nakano, et al., J. Biochem. (Tokyo) 120:803-812, 1996), and circulates at high levels in the bloodstream [Y. Arita, et al., Biochem. Biophys. Res. Commun. 257:79-83, 1999). f-Adiponectin forms oligomers through disulfide bond formation mediated by Cys in the amino terminus and exists as three major oligomeric forms: a trimer, a hexamer and a high molecular weight (HMW) form (U. B. Pajvani, et al., J. Biol. Chem. 278:9073-9085, 2003; K. Kishida, et al., Biochem. Biophys. Res. Commun. 306:286-292, 2003). A proteolytic product of f-adiponectin containing the globular domain (globular adiponectin: g-adiponectin) exists as a trimer (T. S. Tsao, et al., J. Biol. Chem. 277:29359-29362, 2002). Differential biological functions have been reported among the oligomeric forms of f-adiponectin and g-adiponectin.

Adiponectin receptors, AdipoR1 and 2, have been identified in various tissues and cell types [T. Yamauchi, et al., Nature 423:762-769, 2003), although their expression in prostate has not previously been reported. AdipoR1 is abundantly expressed in skeletal muscle, while AdipoR2 is predominant in liver (T. Yamauchi, et al., supra, 2003). These receptors mediate cellular functions of both f- and g-adiponectin via activation of intracellular signaling pathways (T. Yamauchi, et al., supra, 2003). Several signaling molecules, such as 5′-AMP-activated protein kinase (AMPK), nuclear factor (NF)-κB, peroxisome proliferator-activated receptor (PPAR)-α and p38 mitogen-activated protein (MAP) kinase are known to mediate adiponectin-induced metabolic alterations. Some of the signaling pathways are differentially regulated by distinct forms of adiponectin. f-Adiponectin, but not g-adiponectin, down-regulates genes involved in gluconeogenesis through AMPK in liver (T. Yamauchi, et al., Nat. Med. 8:1288-1295, 2002). In contrast, both f- and g-adiponectin stimulate fatty acid oxidation, glucose uptake and lactate production via AMPK activation in C2C12 myocytes (T. Yamauchi, et al., supra, 2002). Interestingly, the trimeric form of f-adiponectin, but not the hexameric or HMW form, activates AMPK in C2C12 cells (T. S. Tsao, et al., J. Biol. Chem. 278:50810-50817, 2003). Activation of NF-κB by adiponectin in C2C12 cells is also oligomerization state-dependent: hexameric and HMW forms of f-adiponectin, but not trimeric f- or g-adiponectin, stimulate NK-κB activation in C2C12 cells (T. S. Tsao, et al., supra, 2003). Adiponectin regulates endothelial cell function as well. Contradictory to the observation that f-adiponectin inhibits angiogenesis through endothelial cell apoptosis in capillary endothelial cells in vitro and in a mouse tumor model in vivo (E. Brakenhielm, et al., Proc. Natl. Acad. Sci. USA 101:2476-2481, 2004), f-adiponectin stimulates angiogenesis via cross-talk between AMPK and Akt in umbilical vein endothelial cells (HUVECS) in vitro (N. Ouchi, et al., J. Biol. Chem. 279:1304-1309, 2004). Furthermore, the HMW form of f-adiponectin selectively suppresses apoptosis with concomitant stimulation of AMPK in HUVECs (H. Kobayashi, et al., Circ. Res. 94:e27-31, 2004]. These contradictions may be due to differences in endothelial cell types used here and in microenvironments between in vivo and in vitro (E. Brakenhielm, et al., supra, 2004). f-Adiponectin also stimulates NO production mediated by Akt through AMPK in bovine aortic endothelial cells (H. Chen, et al., J. Biol. Chem. 278:45021-45026, 2003). In addition, f-adiponectin inhibits tumor necrosis factor-α-induced NF-κB activation through protein kinase A in human aortic endothelial cells (N. Ouchi, et al., Circulation 102:1296-1301, 2000). Although adiponectin has been demonstrated to modulate such signaling pathways, the effect of adiponectin on either c-Jun NH₂-terminal kinase (JNK) or signal transducer and activator of transcription (STAT) pathways has not been reported.

JNK constitutes one of the mammalian mitogen-activated protein (MAP) kinase families and is activated in response to various stimuli, including cytokines [B. Derijard, et al., Cell 76:1025-1037, 1994; M. Hibi, et al., Genes Dev. 7:2135-2148, 1993; J. M. Kyriakis, et al., Nature 369:156-160, 1994; J. Raingeaud, et al., J. Biol. Chem. 270:7420-7426, 1995), and mediates the phosphorylation and activation of such transcription factors as c-Jun (R. J. Davis, et al., Biochem. Soc. Symp. 64:1-12, 1999). JNK is involved in the regulation of cell proliferation and apoptosis during various physiological and pathological events, including tumor development (R. J. Davis, Cell 103:239-252, 2000). In addition, JNK plays a crucial role in obesity and insulin resistance (J. Hirosumi, et al., Nature 420:333-336, 2002; U. Ozcan, et al., Science 306:457-461, 2004).

The transcription factor STAT3 regulates diverse cellular functions, such as cell proliferation, survival, apoptosis and differentiation (D. E. Levy, and J. E. Darnell, Jr., Nat. Rev. Mol. Cell. Biol. 3:651-662, 2002). In response to cytokines and growth factors, such as IL-6 family cytokines and leptin, STAT3 is activated through phosphorylation at Tyr-705 mediated by Janus kinase (JAK). The Tyr-phosphorylation allows STAT3 to dimerize, translocate to the nucleus and activate transcription from target gene promoters containing a sis-inducible element (SIE) (D. S. Aaronson, and C. M. Horvath, Science 296:1653-1655, 2002). Constitutive STAT3 activation is crucial in malignant transformation and cancer progression (T. Bowman, et al., Oncogene 19:2474-2488, 2000). Furthermore, STAT3 is involved in obesity and diabetes (Q. Gao, et al., Proc. Natl. Acad. Sci. USA 101:4661-4666, 2004).

In this study, we determine the effects of f- and g-adiponectin on activation of JNK and STAT3 in prostate cancer DU145, PC-3 and LNCaP-FGC cells, hepatocellular carcinoma HepG2 cells, and C2C12 myoblasts. We show that both f- and g-adiponectin stimulate JNK activation in these cell lines and inhibit STAT3 activation significantly in DU145 and HepG2 cells, in which STAT3 is constitutively activated. This suggests that JNK and STAT3 may be involved in the adiponectin regulation of metabolic disorders and that adiponectin may affect pathogenesis of prostate cancer and hepatocellular carcinoma by acting on tumor cells directly through modulation of these molecules.

Materials and Methods

Cytokines and Antibodies

Recombinant human g-adiponectin was purchased from PeproTech, Inc. (Rocky Hill, N.J.). Recombinant human f-adiponectin was from R & D Systems, Inc. (Minneapolis, Minn.). Velocity sedimentation with sucrose gradients reveled that the batch of human recombinant f-adiponectin used for this study was composed of the HMW and lower molecular weight forms with the majority of the HMW form (data not shown). The stress-activated protein kinase/JNK assay kit, anti-JNK, anti-c-Jun, anti-phospho-c-Jun (Ser-63), and anti-phospho-c-Jun (Ser-73) polyclonal antibodies were obtained from Cell Signaling Technology, Inc. (Beverly, Mass.). The anti-STAT3 antibody for the supershift assay was purchased from Santa Cruz Biotechnology, Inc. (Santa Cruz, Calif.).

Cell Lines and Culture Conditions

Human prostate cancer cell DU145, PC-3 and LNCaP-FGC cells, human hepatocellular carcinoma HepG2 cells, and murine immortalized C2C12 myoblasts were purchased from the American Type Culture Collection (Manassas, Va., USA). DU145, HepG2 and C2C12 cells were cultured in Dulbecco's modified Eagle's medium supplemented with 10% fetal bovine serum (FBS) plus penicillin (100 units/ml) and streptomycin (100 μg/ml). PC-3 and LNCaP-FGC cells were grown in RPMI 1640 supplemented with 10% FBS and antibiotics.

Semi-Quantitative Reverse Transcriptase-PCR Analysis

Total RNA was isolated using the TRIZOL standard technique from DU145, PC-3, LNCaP-FGC and HepG2 cells deprived of serum for 24 hours. cDNA was then generated from total RNA, and reaction mixtures for PCR were prepared as described before (M. Onuma, et al., J. Biol. Chem. 278:42660-42667, 2003). PCR primers to detect AdipoR1 and AdipoR2 are as follows: AdipoR1 (forward: 5′-AGGACAACGACTATCTGCTAC-3′ and reverse: 5′-CATCCCAAAACCACCTTCTC-3′) and AdipoR2 (forward: 5′-AGAGAAAGTGGTGGGGAAAG and reverse: 5′-GGGCGAGGGAGGAAAATAAC-3′). PCR was carried out with an initial denaturing at 94° C. for 1 minute, followed by 23-29 cycles consisting of denaturing at 94° C. for 1 minute, annealing at 55° C. for 1 minute, and extending at 72° C. for 1 minute. Glyceraldehyde-3-phosphate dehydrogenase (GAPDH) was employed as an internal standard to normalize sample variation as described previously (M. Onuma, et al., supra, 2003). PCR products were analyzed by electrophoresis on 2% agarose gels with ethidium bromide staining, and compared after reaction cycles that showed DNA amplification in a linear range.

Cell Lysate Preparation

Seventy-percent confluent cells were deprived of serum for 24 hours, followed by treatment with 1 μg/ml f- or g-adiponectin or 10 μg/ml anisomycin for indicated periods. After being rinsed with cold phosphate-buffered saline, cells were harvested using a scraper and collected by centrifugation at 700×g for 10 minutes at 4° C. The cell pellets were then homogenized in cell lysis buffer (20 mM HEPES, pH 7.9, 300 mM NaCl, 1 mM Na₃VO₄, 1 μg/ml leupeptin, 5 μg/ml aprotinin, 1 μg/ml pepstatin, and 1 mM phenylmethylsulfonyl fluoride) and rocked gently for 30 minutes at 4° C. The homogenates were centrifuged at 15,000×g for 5 minutes at 4° C., and the supernatants were saved as cell lysates and stored at −80° C. until use.

In Vitro JNK Assay

Using the stress-activated protein kinase/JNK assay kit, JNK activity was assessed with a fusion protein of the c-Jun NH₂ terminus (amino acids 1 to 89) and glutathione S-transferase as a substrate according to the manufacture's instructions with some modifications as reported before (M. Onuma, et al., supra, 2003). Briefly, JNK was precipitated in cell lysates (250-500 μg of protein) with 2 μg of glutathione-Sepharose beads immobilized by the fusion protein. The precipitates were rinsed with the cell lysis buffer three times, followed by incubation in 50 μl of the kinase buffer containing 100 μM ATP at 30° C. for 30 minutes. The reaction was terminated by adding the SDS sample buffer. The substrate fusion protein was separated by SDS-polyacrylamide gel electrophoresis, and its phosphorylation by JNK was determined by Western blot analysis using anti-phospho-c-Jun (Ser-63) polyclonal antibody. To normalize sample variations, JNK protein levels were also determined by Western blot analysis using anti-JNK antibody.

Measurement of c-Jun Phosphorylation by Western Blot Analysis

Cell lysates (100 μg of protein) were electrophoresed on 10% SDS-polyacrylamide gels and transferred to polyvinylidene difluoride membranes. After being washed and blocked, the membranes were hybridized with anti-phospho-c-Jun (Ser-63 or Ser-73) polyclonal antibody that specifically detects phosphorylated c-Jun molecules. The membranes were then stripped and re-hybridized with anti-c-Jun antibody that recognizes both phosphorylated and unphosphorylated c-Jun molecules to normalize sample variations.

Electromobility Shift Assay (EMSA)

The EMSA was performed as described previously (Y. Iwamoto, et al., J. Biol. Chem. 273:18198-18204, 1998). Cell lysates containing 10 μg of protein were subjected to the assay with ³²P-end-labeled, double-stranded oligonucleotide M67-SIE (forward: 5′AATTCCATTTCCCGTAAATCCCTG-3′ and reverse: 5′-AATTCAGGGATTTACGGGAAATGG-3′) as a probe. The supershift assay was carried out to confirm the STAT3-DNA complex using anti-STAT3 antibody.

Results

Expression of AdipoR1 and AdipoR2 by Prostate Cancer and Hepatocellular Carcinoma Cells

The adiponectin receptors AdipoR1 and AdipoR2 have been identified in various tissues and cell types, including myocytes and hepatocytes (T. Yamauchi, et al., Nature 423:762-769, 2003). However, their expression by cancer cells has not been reported.

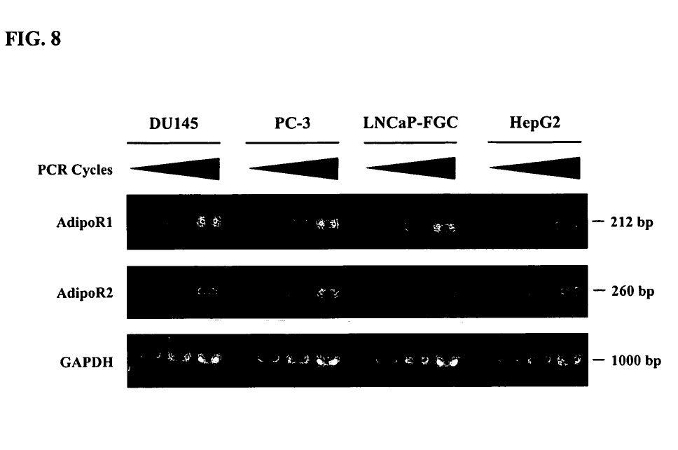

Using quantitative RT-PCR, we profiled expression of AdipoR1 and AdipoR2 in prostate cancer DU145, PC-3 and LNCaP-FGC cells, and hepatocellular carcinoma HepG2 cells (FIG. 8). All four cell lines expressed mRNA of both receptors. AdipoR1 and AdipoR2 were both expressed at similar levels in the three prostate cancer cell lines. HepG2 cells expressed more AdipoR2 and less AdipoR1 than prostate cancer cells. This is consistent with a previous report that AdipoR2 is the predominant form expressed in liver (T. Yamauchi, et al., supra, 2003).

Stimulation of JNK Activation by f- and g-Adiponectin

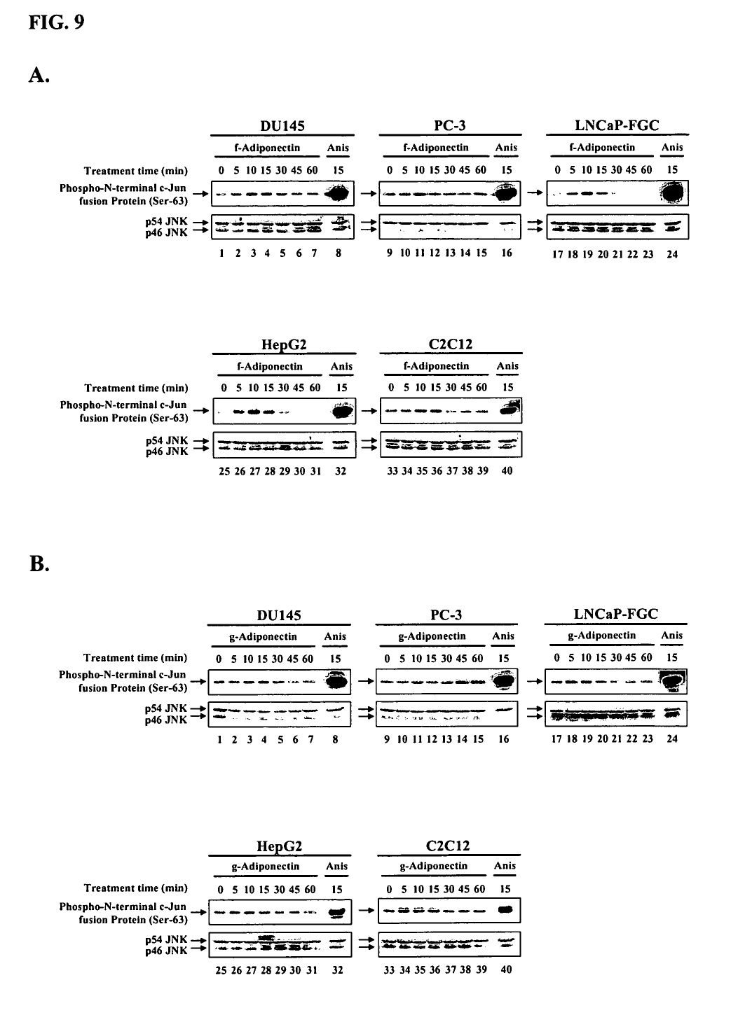

We examined JNK activity in prostate cancer DU145, PC-3 and LNCaP-FGC cells, hepatocellular carcinoma HepG2 cells and C2C12 myoblasts treated with f- or g-adiponectin for periods up to 60 minutes. JNK was constitutively activated at low levels in all the five cell lines, and f-adiponectin further stimulated JNK activation, peaking at 10 minutes after addition of f-adiponectin (FIG. 9A). Likewise, g-adiponectin stimulated JNK activation in all the cell lines (FIG. 9B). However, g-adiponectin maximized JNK activation at different time points varying between 5 and 15 minutes after cytokine addition among the cell lines.

Stimulation of c-Jun Phosphorylation by f- and g-Adiponectin

c-Jun is a physiological substrate for JNK (E. Shaulian, and M. Karin, Nat. Cell Biol. 4:E131-136, 2002). JNK phosphorylates c-Jun at Ser-63 and Ser-73, and phosphorylation of these residues is required for c-Jun activation (E. Shaulian, and M. Karin, supra, 2002).

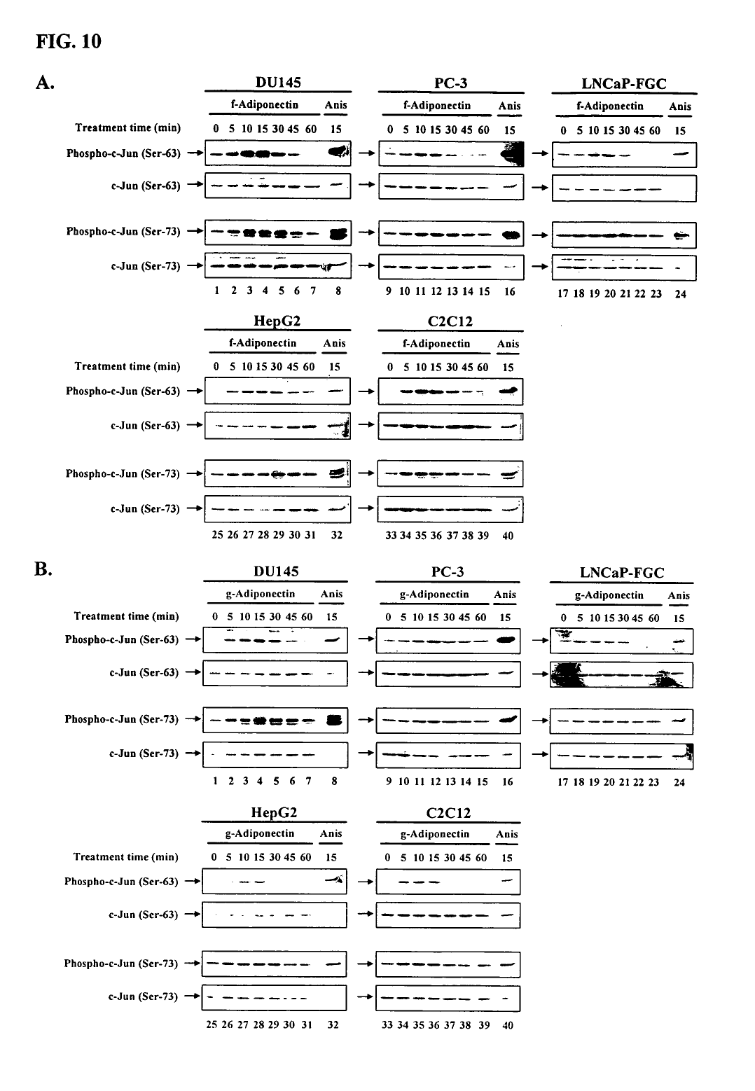

We examined c-Jun phosphorylation during f- or g-adiponectin treatment in DU145, PC-3, LNCaP-FGC, HepG2 and C2C12 cells. c-Jun was constitutively phosphorylated at Ser-63 and Ser-73 in all the five cell lines. f- and g-Adiponectin both augmented c-Jun phosphorylation at Ser-63 in the five cell lines (FIGS. 10A and 10B) in correlation with JNK activation (FIGS. 9A and 9B). Both forms of adiponectin also stimulated Ser-73 phosphorylation in all the cell lines although the stimulation was quite modest in LNCaP-FGC cells (FIGS. 10A and 10B). It should be noted that adiponectin induced Ser-63 phosphorylation to a higher extent than Ser-73 phosphorylation and that adiponectin stimulated Ser-73 phosphorylation longer than Ser-63 phosphorylation. This indicates that Ser-63 is more susceptible to both phoshporylation and dephosphorylation than Ser-73 during adiponectin treatment. Therefore, Ser-63 phoshporylation may play a major role in the immediate control of c-Jun activation by adiponectin, and Ser-73 phosphorylation may be important in the sustained activation of c-Jun.

Inhibition of Constitutive STAT3 Activation by f- and g-Adiponectin

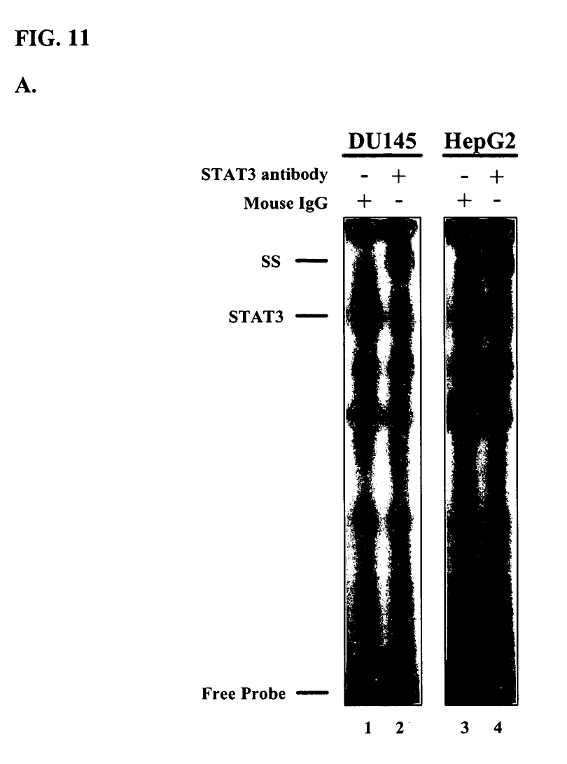

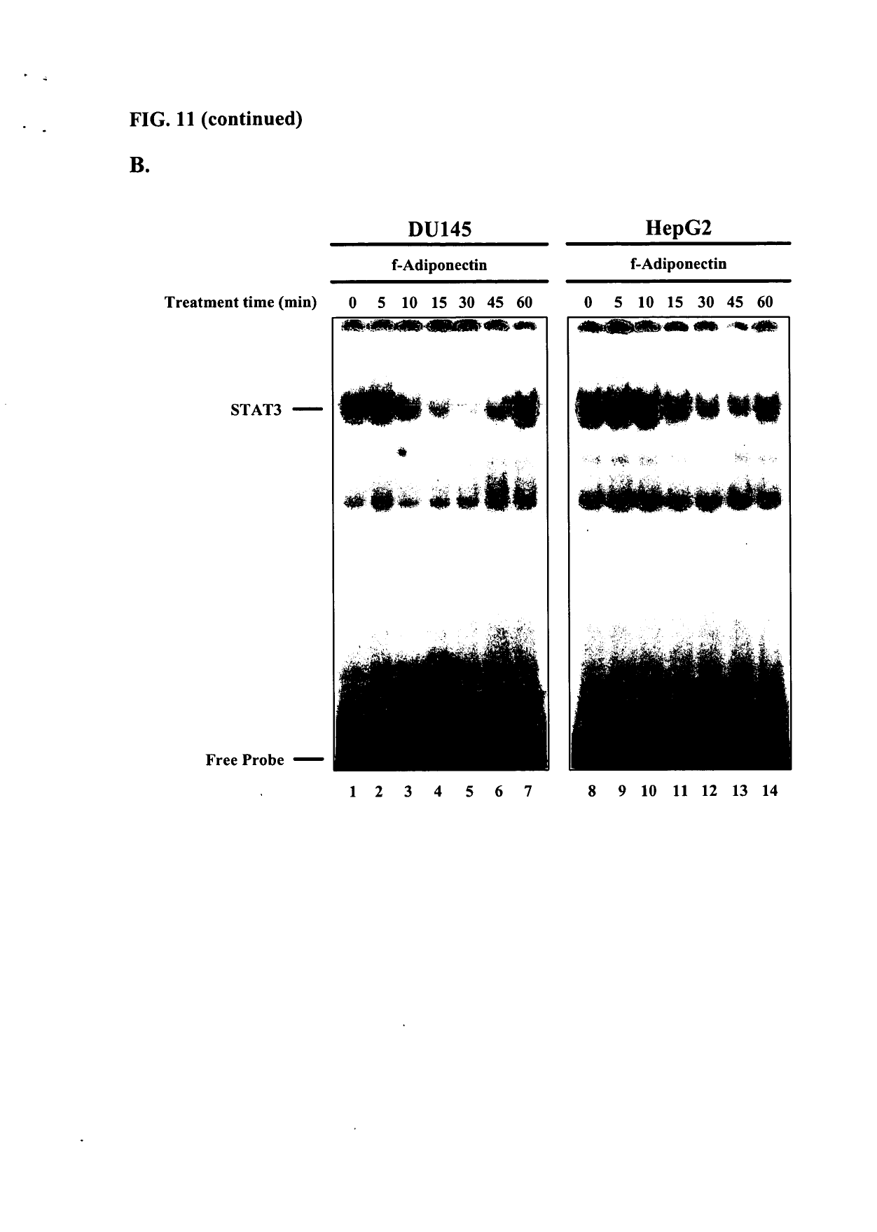

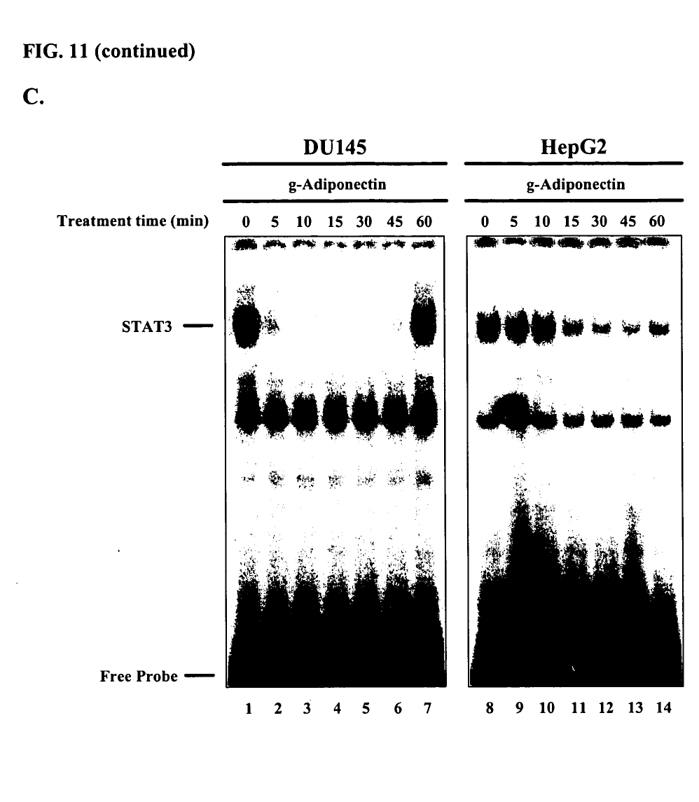

We examined the effect of f- and g-adiponectin on STAT3 activation using the EMSA. Constitutive STAT3 activation was detected in DU145 and HepG2 cells (FIG. 11A). Both f- and g-adiponectin inhibited constitutive STAT3 activation drastically in these cell lines (FIGS. 11B and 11C). STAT3 activation returned to the original level in DU145 cells and began to rise in HepG2 cells within 60 minutes after adding f- or g-adiponectin. Neither f- nor g-adiponectin influenced STAT3 activation in any of the other cell lines (data not shown).

Discussion

Adiponectin is a major adipose cytokine that ameliorates metabolic disorders, such as obesity and diabetes mellitus (J. Fruebis, et al., supra, 2001; A. H. Berg, et al., supra, 2001). Adiponectin may also influence cancer pathogenesis (T. Yokota, et al., supra, 2000; E. Brakenhielm, et al., supra, 2004). Obesity is associated with certain types of cancer, including prostate cancer (C. Rodriguez, et al., supra, 2001; C. L. Amling, et al., supra, 2001; Y. Furuya, et al., supra, 1998) and hepatocellular carcinoma (J. M. Regimbeau, et al., Liver Transpl. 10:S69-73, 2004]. In this study, we employed prostate cancer cells, hepatocellular carcinoma cells and immortalized myoblasts, and showed for the first time that adiponectin modulates activation of the JNK and STAT3 pathways. This suggests that JNK and STAT3 may be involved in adiponectin-mediated, metabolic alteration and that adiponectin may influence pathophysiology of these obesity-associated cancers by acting on tumor cells directly mediated by these signaling pathways.

Both f-adiponectin (consisting mainly of the HMW form) and g-adiponectin stimulated JNK activation (FIG. 9) and c-Jun phosphorylation (FIG. 10) in all the cell lines examined, and inhibited STAT3 activation where it was constitutively activated in DU145 and HepG2 cells (FIG. 11). As mentioned above, extensive evidence indicates that distinct forms of adiponectin differentially activate such signaling molecules as AMPK and NF-κB to mediate diverse biological functions among different cell types. Therefore, JNK and STAT 3 may be involved in pivotal pathways for any forms of adiponectin in universal biological functions, while such signaling pathway as AMPK and NF-κB pathways may determine specific roles of distinct forms of adiponectin in certain functions.

Adiponectin exerts antidiabetic effects by enhancing insulin action through such signaling pathways as the AMPK pathway in muscle and liver as stated above (A. H. Berg, et al., supra, 2001; T. Yamauchi, et al., supra, 2003; T. Yamauchi, et al., supra, 2002). We demonstrated that adiponectin activated JNK remarkably in C2C12 myoblasts and hepatocellular carcinoma HepG2 cells (FIG. 9), suggesting the possible involvement of JNK in adiponectin-mediated, antidiabetic effects. It is interesting to note that JNK activity is abnormally elevated in muscle, liver and adipose tissue in obesity and insulin resistance (J. Hirosumi, et al., supra, 2002). Highly activated JNK in obesity may mediate a negative feedback loop to maintain normal metabolic homeostasis by adiponectin: activated JNK may sensitize muscle cells and hepatocytes to adiponectin, enhancing its antidiabetic effects. Furthermore, JNK activation accompanied STAT3 inhibition in HepG2 cells during adiponectin treatment (FIG. 11). Thus, STAT3 inhibition concomitant with JNK activation may be important in antidiabetic action of adiponectin in hepatocytes.

JNK plays a crucial role in various cancers as well (R. J. Davis, et al., supra, 2000). We demonstrated that adiponectin stimulated JNK activation (FIG. 9) and c-Jun phosphorylation (FIG. 10) in all the four cancer cell lines examined, including prostate cancer DU145, PC-3 and LNCaP-FGC cells and hepatocellular carcinoma HepG2 cells (FIGS. 9 and 10). Furthermore, adiponectin inhibited STAT3 activation in DU145 and HepG2 cells (FIGS. 11B and 11C), in which STAT3 was constitutively activated (FIG. 11A). Therefore, adiponectin acts on these cancer cells directly and may regulate their function through modulation of JNK and STAT3. It should be noted that constitutive STAT3 activation induces malignant transformation and stimulates cancer progression including cell growth promotion (T. Bowman, et al., supra, 2000). Thus, inhibition of constitutive STAT3 activation is considered to be a therapeutic intervention for cancer (T. Bowman, et al., supra, 2000). Therefore, adiponectin may also be therapeutic through STAT3 inactivation. Further investigation is ongoing in our laboratory to address potential roles of adiponectin in cancer pathogenesis.

In conclusion, JNK and STAT3 may play a role in adiponectin suppression of metabolic syndrome, and adiponectin may influence pathogenesis of prostate cancer and hepatocellular carcinoma by acting on tumor cells directly via these signaling molecules. Since obesity is associated with some types of cancer, including prostate cancer (C. Rodriguez, et al., supra, 2001; C. L. Amling, et al., supra, 2001; Y. Furuya, et al., supra, 1998) and hepatocellular carcinoma (J. M. Regimbeau, et al., supra, 2004), adiponectin, as well as JNK and STAT3, may be molecular mediators between obesity and its associated cancers and could be their common therapeutic targets.

C. Adiponectin as a Growth Inhibitor in Prostate Cancer Cell Growth

Abstract

Prostate cancer, the second leading cause of cancer death among American, is associated with obesity, another serious health problem in the United States. However, the molecular basis of this association is not well known. We have previously reported that adipose cytokines leptin and IGF-I, whose blood levels are elevated in obesity, stimulate androgen-independent prostate cancer cell growth synergistically. Adiponectin is another major adipose cytokine that decreases in circulation in obesity and ameliorates obesity. Here we identify adiponectin as a novel growth inhibitor in prostate cancer cell growth. Adiponectin occurs in non-proteolytic (full-length adiponectin: f-adiponectin) and proteolytic (globular adiponectin) forms in various oligomeric states (trimer, hexamer, and high molecular weight complex). Distinct forms of adiponectin exert differential biological effects. The 3-(4,5-Dimethylthiazol-2-yl)-2,5-diphenyl tetrazolium bromide assay demonstrates that f-adiponectin inhibits prostate cancer cell growth drastically at subphysiological concentrations in androgen-independent DU145 and PC-3 and androgen-dependent LNCaP-FGC cells. Furthermore, velocity sedimentation analysis shows that the high molecular weight complex of f-adiponectin is the inhibitory form. Moreover, f-adiponectin suppresses leptin- and/or IGF-I-stimulated, androgen-independent DU145 cell growth, as well as dihydrotestosterone-stimulated, androgen-dependent LNCaP-FGC cell growth. In addition, f-adiponectin enhances doxorubicin inhibition of prostate cancer cell growth. These findings suggest that f-adiponectin is a molecular mediator between prostate cancer and obesity, and may be therapeutic to both androgen-independent and -dependent prostate cancer: f-adiponectin could be used as an adjuvant for cytotoxic chemotherapy or androgen ablation therapy; and reducing obesity may slow androgen-independent and -dependent prostate cancer progression through increasing f-adiponectin production by adipocytes.

Introduction

Prostate cancer is the second leading cause of cancer death among American men. Despite its relatively slow progression, many patients have recurrent disease. Initial treatment for patients with recurrence is androgen ablation therapy to block function of androgen as a potential growth stimulant for prostate cancer. Even though this approach is initially effective in the majority of patients, eventually the disease becomes androgen-independent, returns, and culminates in the patients' death. Therefore, it is desired to develop effective therapies and preventives for androgen-independent prostate cancer (AIPC).

Obesity is another serious health problem in the United States. Intriguingly, prostate cancer, especially its accelerated progression (Amling, C. L., et al., supra, 2001; Furuya, Y., et al., supra, 1998; Rodriguez, C., et al., supra, 2001), is associated with obesity (adiposity). However, little is understood about this association at a molecular level.

Adipose tissue is widely recognized as an endocrine organ, and adipocytes secrete various factors, including cytokines and hormones, that play crucial roles in obesity (Fruhbeck, G., et al., Am. J. Physiol. Endocrinol. Metab. 280: E827-847, 2001). We have demonstrated that conditioned medium from adipocyte culture augments prostate cancer cell proliferation (Onuma, M., et al., J. Biol. Chem. 278:42660-42667, 2003), indicating that adipose factors stimulate prostate cancer cell growth.

Leptin, one of the major adipose cytokines, controls body weight through food intake and energy expenditure (Campfield, L. A., et al., supra, 1995; Pelleymounter, M. A., et al., Science 269:540-543, 1995). Adipose tissue leptin mRNA and circulating leptin levels (Maffei, M., et al., Nat. Med. 1:1155-1161, 1995; Dagogo-Jack, S., et al., Diabetes 45:695-698, 1996; Haffner, S. M., et al., Int. J. Obes. Relat. Metab. Disord. 20:904-908, 1996; Ostlund, R. E., et al., J. Clin. Endocrinol. Metab. 81:3909-3913, 1996) increase in obesity. Clinical studies demonstrate that blood leptin levels may affect the risk of clinically relevant prostate cancer (Chang, S., et al., Prostate 46:62-67, 2001; Stattin, P., et al., supra, 2001) although some controversies exist (Lagiou, P., et al., Int. J. Cancer 76:25-28, 1998; Hsing, A. W., et al., J. Natl. Cancer Inst. 93:783-789, 2001; Stattin, P., et al., Cancer Epidemiol. Biomarkers Prev. 12:474-475, 2003). Of interest, leptin has been identified as a growth factor in AIPC cell growth: leptin stimulates cell proliferation in androgen-independent DU145 and PC-3 cells (Onuma, M., et al., supra, 2003; Somasundar, P., et al., J Surg. Res. 118:71-82, 2004) but not in androgen-dependent LNCaP-FGC cells (Onuma, M., et al., supra, 2003). Leptin is not the only adipose cytokine that plays a role both in body weight homeostasis and AIPC cell growth. Adipocytes secrete insulin-like growth factor-I (IGF-I) (Fruhbeck, G., et al., supra, 2001), too. IGF-I is implicated in energy homeostasis (Kaaks, R. and Lukanova, Proc. Nutr. Soc. 60:91-106, 2001), and circulating levels of free IGF-I are elevated in obesity (Frystyk, J., et al., Metabolism 44:37-44, 1995; Nam, S. Y., et al., Int. J. Obes. Relat. Metab. Disord. 21: 355-359, 1997). Interestingly, as with leptin, plasma IGF-I levels are associated with prostate cancer risk (Chan, J. M., et al., supra, 1998) and may predict development of advanced-stage prostate cancer (Chan, J. M., et al., supra, 2002). IGF-I stimulates cell proliferation in the absence of androgen in AIPC cells but not in androgen-dependent cells (Onuma, M., et al., supra, 2003; Iwamura, M., et al., supra, 1993). Interleukin-6 (IL-6) is also secreted by adipocytes (Fruhbeck, G., et al., supra, 2001) in addition to other cell types and involved in mature-onset obesity (Wallenius, V., et al., Nat. Med. 8:75-79, 2002). IL-6 production by adipose tissue, as well as blood IL-6 levels, increases in obesity (Mohamed-Ali, V., et al., J. Clin. Endocrinol. Metab. 82:4196-4200, 1997; Kern, P. A., et al., Am. J. Physiol. Endocrinol. Metab. 280:E745-751, 2001). Serum levels of IL-6 are associated with clinically evident hormone-resistant prostate cancer (Drachenberg, D. E., et al., supra, 1999). IL-6 stimulates cell proliferation in AIPC cells but inhibits in androgen-dependent cells (Onuma, M., et al., supra, 2003; Chung, T. D., et al., supra, 1999; Okamoto, M., et al., supra, 1997). Intriguingly, leptin interacts with IGF-I and IL-6 to promote AIPC cell proliferation (Onuma, M., et al., supra, 2003). Therefore, adipose cytokines, which play a role in metabolic syndrome and whose production is augmented in obesity, are likely to stimulate AIPC cell growth.

Adiponectin is a cytokine that is expressed most abundantly and specifically in adipose tissue (Maeda, K., et al., Biochem. Biophys. Res. Commun. 221:286-289, 1996; Hu, E., et al., J. Biol. Chem. 271:10697-10703, 1996; Scherer, P. E., et al., J. Biol. Chem. 270:26746-26749, 1995). This approximately 30-kDa polypeptide called full-length adiponectin (f-adiponectin) consists of an amino-terminal signal sequence, a variable domain, a collagen-like domain and a carboxyl-terminal globular domain (Maeda, K., et al., supra, 1996; Hu, E., et al., supra, 1996; Scherer, P. E., et al., supra, 1995; Nakano, Y., et al., J. Biochem. (Tokyo) 120:803-812, 1996), and circulates at high levels in the bloodstream (Arita, Y., et al., Biochem. Biophys. Res. Commun. 257:79-83, 1999). f-Adiponectin forms oligomers through disulfide bond formation mediated by Cys in the amino terminus and exists as three major oligomeric forms in circulation: trimeric, hexameric and high molecular weight (HMW) forms (Pajvani, U. B., et al., J. Biol. Chem. 278:9073-9085, 2003; Kishida, K., et al., Biochem. Biophys. Res. Commun. 306:286-292, 2003). A proteolytic product of f-adiponectin containing the globular domain (referred to as globular adiponectin: g-adiponectin) is also biologically active and circulates at low levels (Fruebis, J., et al., Proc. Natl. Acad. Sci. USA 98:2005-2010, 2001). g-Adiponectin exists as a trimer (Tsao, T. S., et al., J. Biol. Chem. 277:29359-29362, 2002). Differential biological activities and signaling properties have been reported among the oligomeric forms of f-adiponectin, and g-adiponectin (Tsao, T. S., et al., supra, 2002; Yamauchi, T., et al., Nat. Med. 8:1288-1295; 2002; Tsao, T. S., et al., J. Biol. Chem. 278:50810-50817, 2003; Kobayashi, H., et al., Circ. Res. 94:e27-31, 2004).

AdipoR1 and 2, cell surface receptors for adiponectin, are found in a variety of tissue and cell types (Yamauchi, T., et al., Nature 423:762-769, 2003). AdipoR1 is abundantly expressed in skeletal muscle, and AdipoR2 is predominant in liver (Yamauchi, T., et al., supra, 2003). We have recently shown that prostate cancer cells express both receptors (Miyazaki, T., et al., Biochem. Biophys. Res. Commun. 333:79-87, 2005 (in press)). These receptors mediate cellular functions of f- and g-adiponectin (Yamauchi, T., et al., supra, 2003) through modulation of diverse signaling molecules, including 5′-AMP-activated protein kinase (Yamauchi, T., et al., supra; 2002; Tsao, T. S., et al., supra, 2003; Kobayashi, H., et al., supra, 2004; Ouchi, N., et al., J. Biol. Chem. 279:1304-1309, 2004; Chen, H., et al., J. Biol. Chem. 278:45021-45026, 2003), nuclear factor-KB (Tsao, T. S., et al., supra, 2002; Tsao, T. S., et al., supra, 2003; Ouchi, N., et al., Circulation 102:1296-1301, 2000), Akt (Ouchi, N., et al., supra, 2004; Chen, H., et al., supra, 2003), peroxisome proliferator-activated receptor-α (Yamauchi, T., et al., supra, 2003), p38 mitogen-activated protein kinase (Yamauchi, T., et al., supra, 2003), protein kinase A (Ouchi, N., et al., supra, 2000), c-Jun NH₂-terminal kinase (Miyazaki, T., et al., supra, 2005), and signal transducer and activator of transcription 3 (STAT3) (Miyazaki, T., et al., supra, 2005).

In contrast to leptin, plasma levels of adiponectin negatively correlate with body mass index (Arita, Y., et al., supra, 1999) and visceral fat accumulation (Cnop, M., et al., Diabetologia 46:459-469, 2003). Furthermore, low levels of plasma adiponectin are associated with such obesity-related disorders as diabetes mellitus (Hotta, K., et al., Arterioscler. Thromb. Vasc. Biol. 20:1595-1599, 2000) and coronary artery (atherosclerotic) disease (Kumada, M., et al., Arterioscler. Thromb. Vasc. Biol. 23:85-89, 2003). Animal model studies have indicated crucial roles of adiponectin in pathogenic alterations of these disorders. g-Adiponectin treatment increases fatty acid oxidation and causes weight loss in mice fed a high fat/sucrose diet, accompanied by decrease in blood levels of glucose, free fatty acids and triglycerides (Fruebis, J., et al., supra, 2001). An injection of f-adiponectin abolishes hyperglycemia in diabetic mice (ob/ob, NOD (non-obese diabetic) or streptozotocin-treated mice) by enhancing hepatic insulin action (Berg, A. H., et al., Nat. Med. 7:947-953, 2001). Moreover, adiponectin knockout mice demonstrate diet-induced insulin resistance (Maeda, N., et al., supra, 2002) and are susceptible to obesity, hyperglycemia and hypertension as compared to wild-type mice when fed an atherogenic diet (Ouchi, N., et al., Hypertension 42:231-234, 2003). In addition to obesity and its related diseases, several lines of evidence indicate that adiponectin may influence cancer pathogenesis. Low serum adiponectin levels are associated with an increased risk of breast cancer (Mantzoros, C., et al., J. Clin. Endocrinol. Metab. 89:1102-1107, 2004; Miyoshi, Y., et al., Clin. Cancer Res. 9:5699-5704, 2003), and tumors arising in women with low adiponectin levels are more likely to show a biologically aggressive phenotype (Miyoshi, Y., et al., supra, 2003). Circulating adiponectin levels are inversely associated with endometrial cancer risk as well (Petridou, E., et al., J. Clin. Endocrinol. Metab. 88:993-997, 2003; Dal Maso, L., et al., J. Clin. Endocrinol. Metab. 89:1160-1163, 2004). It is interesting to note that, consistent with the inverse association of serum adiponectin levels with obesity, these cancers are associated with obesity (Rose, D. P., et al., Obes. Rev. 5:153-165, 2004; Kaaks, R., et al., Cancer Epidemiol. Biomarkers Prev. 11:1531-1543, 2002). Furthermore, adiponectin has been shown to inhibit cell proliferation and induce apoptosis in leukemia cells in vitro (Yokota, T., et al., Blood 96:1723-1732, 2000) and to suppress tumor growth in mice most likely due to inhibition of neovascularization through suppression of endothelial cell proliferation, migration and survival (Brakenhielm, E., et al., Proc. Natl. Acad. Sci. USA 101:2476-2481, 2004). However, direct effect of adiponectin on solid tumor cell growth has not been reported.

In this study, we identify adiponectin as a novel growth inhibitor in prostate cancer cell growth. Using DU145 and PC-3 cells as models for AIPC cells and LNCaP-FGC cells for androgen-dependent prostate cancer cells, we demonstrate that f-adiponectin inhibits prostate cancer cell growth at subphysiological concentrations in all three cell lines and that the HMW form is the inhibitory oligomer. Furthermore, f-adiponectin suppresses leptin and/or IGF-I-stimulated, androgen-independent cell growth in AIPC cells, as well as dihydrotestosterone (DHT)-stimulated, androgen-dependent cell growth in LNCaP-FGC cells. In addition, f-adiponectin enhances doxorubicin-induced cell growth suppression in the three cell lines. Therefore, f-adiponectin is a growth inhibitor of prostate cancer cells and could be therapeutic for both androgen-independent and -dependent prostate cancer.

Materials and Methods

Cytokines and Antibodies

Recombinant human f-adiponectin was purchased from BioVendor Laboratory Medicine, Inc. (Brno, Czech Republic) and R&D Systems Inc. (Minneapolis, Minn.). Recombinant human leptin and IGF-I were from R&D Systems Inc. Recombinant human g-adiponectin was purchased from PeproTech (Rocky Hill, N.J.). The anti-adiponectin antibody was from Chemicon International Inc. (Temecula, Calif.).

Prostate Cancer Cells and Culture Conditions

Human prostate cancer cell lines DU145, PC-3, and LNCaP-FGC were purchased from the American Type Culture Collection (Manassas, Va.). DU145 cells were cultured in Dulbecco's modified Eagle's medium supplemented with 10% fetal bovine serum (FBS) plus penicillin (100 units/ml) and streptomycin (100 μg/ml). PC-3 and LNCaP-FGC cells were cultured in RPMI 1640 supplemented with 10% FBS and antibiotics.

Preparation of Conditioned Media from Preadipocyte and Mature Adipocyte Cultures

Human primary preadipocytes were purchased from BioWhittaker Inc. (Walkersville, Md.). Preadipocytes were differentiated into mature adipocyte in vitro, conditioned media were prepared from both cell types as described (Onuma, M., et al., supra, 2003).

### 3-(4,5-Dimethylthiazol-2-yl)-2,5-diphenyl Tetrazolium Bromide (MTT) Assay

DU145 (0.5-1.0×10³/well), PC-3 (0.5-1.0×10³/well), and LNCaP FGC (0.75-1.5×10³/well) cells were seeded in 96-well plates 24 hours prior to serum starvation. Cells were starved of serum for 24 hours and then incubated in the serum-free medium with or without cytokine(s), DHT and/or doxorubicin at indicated concentrations for 5 days. MTT was added to each well to a concentration of 0.5 mg/ml. After being incubated at 37° C. for 3 hours, cells were lysed in 50% dimethylformamide and 20% SDS at 37° C. Optical densities (OD) at 550 and 670 nm were measured by a plate reader, and differential OD between 550 and 670 nm (OD 550 minus OD 670 nm) were determined. Each experiment was performed in quadruplicate, and the values were reported as mean±S.D.

Velocity Sedimentation

Sucrose gradients were set up in 2.2-ml centrifuge tubes (Beckman Coulter, Inc., Fullerton, Calif.) by layering 5, 10, 15 and 20% (w/v) sucrose in velocity sedimentation buffer (10 mM HEPES, pH 8; 125 mM NaCl). Gradients were then equilibrated overnight at 4° C. Twenty micrograms of f-adiponectin in 200 μl of velocity sedimentation buffer containing 5 μl of HMW-Native protein marker kit (GE Healthcare, Piscataway, N.J.) set to 0.5 μg/μl albumin was overlayed onto the sucrose gradients and spun in a Beckman TLS-55 rotor at 55,000 rpm for 5 hours at 4° C. Fractions (150 μl each) were sequentially collected from the top to the bottom; aliquots were removed and used immediately in the MTT assay. The remaining material was stored at −80° C. for Western blot analysis with anti-adiponectin antibody and SDS-PAGE with silver staining. For the MTT assay, DU145 cells (0.5×1 03/well) were serum-starved and incubated in triplicate in 60 μl of the serum-free medium plus 30 μl of each fraction. For Western blot analysis, 0.5 μl of each fraction was electrophoresed on a 10% SDS-polyacrylamide gel and electrotransferred to a polyvinylidene difluoride membrane. The membrane was washed, blocked, and hybridized with the anti-adiponectin antibody. Bands were visualized using a Lumiglo reagent (Cell Signaling Technology, Inc., Beverly, Mass.). For SDS-PAGE, 20 μl of each fraction was electrophoresed on a 10% SDS-polyacrylamide gel, followed by silver staining using the Silver Stain kit (Bio-Rad Laboratories, Inc., Hercules, Calif.) to visualize molecular weight markers.

Results

f-Adiponectin, but not g-Adiponectin, Inhibits Prostate Cancer Cell Growth at Subphysiological Concentrations.

Adiponectin has been shown to regulate cell growth in some cell types, including leukemia cells (Yokota, T., et al., supra, 2000) and vascular endothelial cells (Kobayashi, H., et al., supra, 2004; Brakenhielm, E., et al., Proc. Natl. Acad. Sci. USA 101:2476-2481, 2004). f- and g-Adiponectin show differential effects on cell growth regulation (Kobayashi, H., et al., supra, 2004).

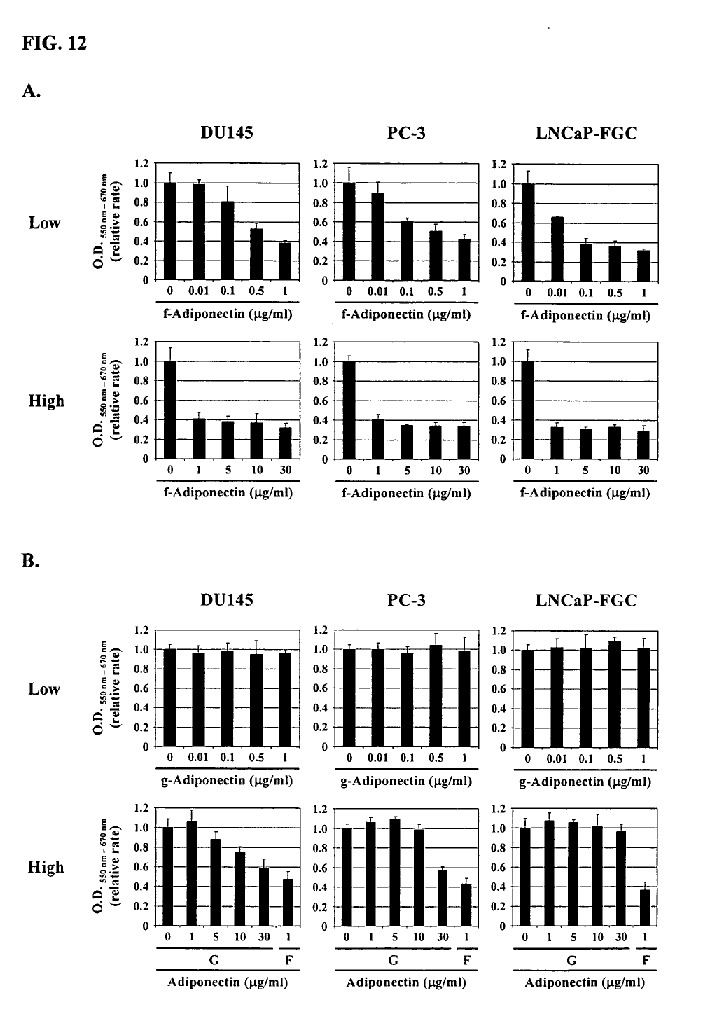

We employed the MTT assay to determine whether f- or g-adiponectin influences cell growth (cell viability) in androgen-independent prostate cancer DU145 and PC-3, and androgen-dependent LNCaP-FGC cells. f-Adiponectin inhibited cell growth in a dose-dependent manner with the inhibitory effect saturated at 1 μg/ml in all the three cell lines (FIG. 12A). In contrast, g-adiponectin did not affect cell growth at concentrations up to 1 μg/ml in any of the prostate cancer cell lines and inhibited at higher concentrations (1-30 μg/ml) in DU145 and PC-3, but not LNCaP-FGC, cells (FIG. 12B). Therefore, f-adiponectin is more inhibitory to prostate cancer cell growth than g-adiponectin.

The HMW Form of f-Adiponectin Inhibits Prostate Cancer Cell Growth.

f-Adiponectin exists as three major oligomeric forms: a trimer, a hexamer, and a HMW form (Pajvani, U. B., et al., supra, 2003; Kishida, K., et al., supra, 2003). Distinct oligomers exert differential biological functions (Tsao, T. S., et al., supra, 2002; Yamauchi, T., et al., supra; 2002; Tsao, T. S., et al., supra, 2003; Kobayashi, H., et al., supra, 2004).

To determine which oligomeric form(s) of f-adiponectin inhibits prostate cancer cell growth, we subjected different lots of f-adiponectin from commercial sources to velocity sedimentation to fractionate oligomers and tested the effects of fractions on DU145 cell growth using the MTT assay. Velocity sedimentation demonstrated variation in the ratios of contained oligomeric forms from one lot to another (data not shown). FIG. 13A shows an oligomeric profile of a lot that contained the HMW form at a high ratio. The same fractions were applied to the MTT assay with corresponding fractions from f-adiponectin-free velocity sedimentation as controls (FIG. 13B). Control fractions suppressed basal levels of cell growth as sucrose concentrations increased. As compared to the controls, the HMW form, but not lower molecular weight forms, inhibited cell growth in DU145 cells (FIG. 13B). We used this lot as f-adiponectin for all the other experiments in this study.

f-Adiponectin Inhibits Stimulatory Adipose Cytokine-Induced, AIPC Cell Growth.

Conditioned medium from adipocyte culture promotes cell growth in prostate cancer cells, indicating that adipocytes secrete factors that stimulate prostate cancer cell growth (Onuma, M., et al., supra, 2003). Stimulatory adipose factors include such cytokines as leptin (Onuma, M., et al., supra, 2003; Somasundar, P., et al., supra, 2004) and IGF-I (Onuma, M., et al., supra, 2003, Iwamura, M., et al., supra, 1993) whose adipocyte production and blood levels are elevated in obesity (Maffei, M., et al., supra, 1995; Dagogo-Jack, S., et al., supra, 1996; Haffner, S. M., et al., supra, 1996; Ostlund, R. E., et al., supra, 1996; Frystyk, J., et al., supra, 1995; Nam, S. Y., et al., supra, 1997). In contrast to these cytokines, another adipose cytokine f-adiponectin, which decreases in circulation in obesity (Arita, Y., et al., supra, 1999; Cnop, M., et al., supra, 2003), inhibits prostate cancer cell growth (FIG. 12). This leads to a hypothesis: f-adiponectin antagonizes stimulatory adipose cytokines to suppress prostate cancer cell growth.

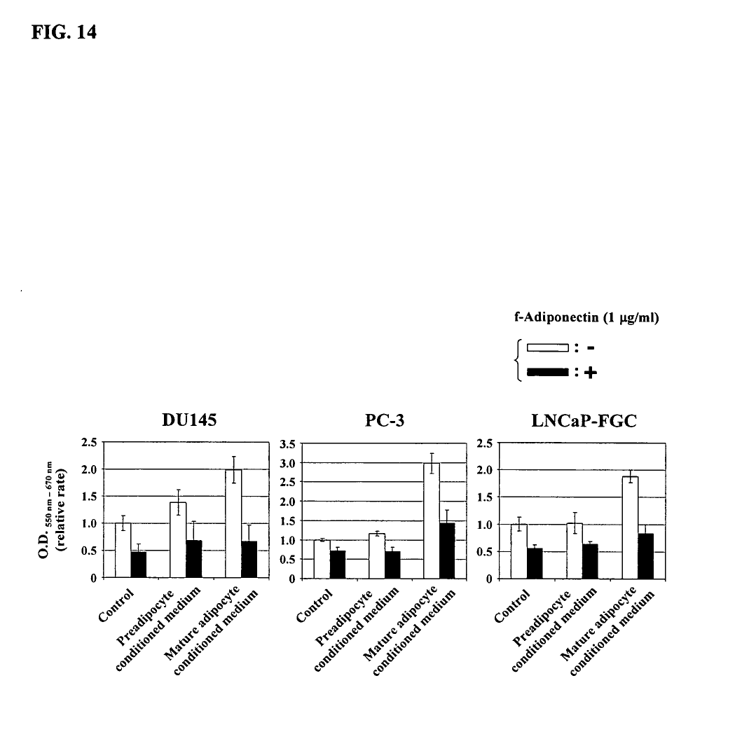

To address this hypothesis, we first examined the effect of f-adiponectin on adipocyte conditioned medium-stimulated cell growth in DU145, PC-3, and LNCaP-FGC cells. Consistent with the previous report (Onuma, M., et al., supra, 2003), the MTT assay demonstrated that adipocyte conditioned medium stimulated cell growth in all the three cell lines (FIG. 14). Amazingly, 1 μg/ml f-adiponectin drastically suppressed the conditioned medium stimulation of prostate cancer cell growth in these cells (FIG. 14). This suggests that f-adiponectin may compete with stimulatory adipose factors to inhibit prostate cancer cell growth.

Of interest, stimulatory adipose cytokines leptin and IGF-I stimulate cell growth in an androgen-independent manner in only androgen-independent, but not androgen-dependent, prostate cancer cells (Onuma, M., et al., supra, 2003; Iwamura, M., et al., supra, 1993), while adipose factors are not known that stimulate androgen-dependent cells.

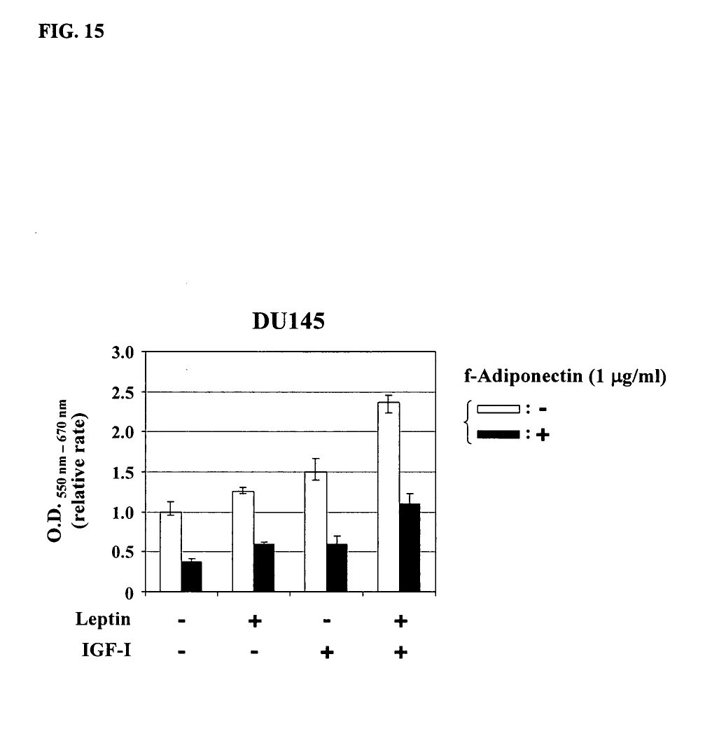

We carried out the MTT assay with androgen-independent DU145 cells to determine the effect of f-adiponectin on leptin and/or IGF-I-stimulated, AIPC cell growth. As previously reported (Onuma, M., et al., supra, 2003), 12.5 μg/ml leptin and 100 ng/ml IGF-I stimulated cell growth synergistically in the absence of androgen (FIG. 15). Strikingly, 1 μg/ml f-adiponectin remarkably suppressed the leptin and/or IGF-I stimulation of prostate cancer cell growth (FIG. 15). Therefore, f-adiponectin antagonizes leptin and IGF-I to suppress AIPC cell growth.

f-Adiponectin Inhibits DHT-Stimulated, Androgen-Dependent Prostate Cancer Cell Growth.

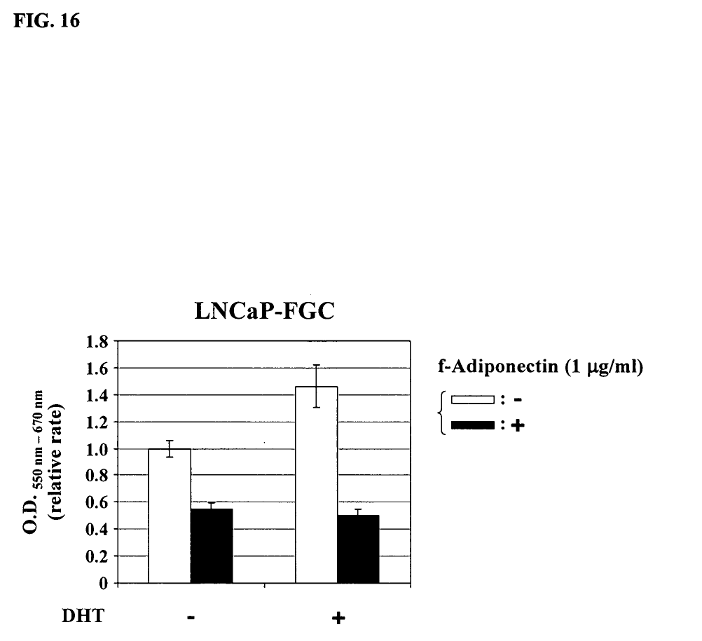

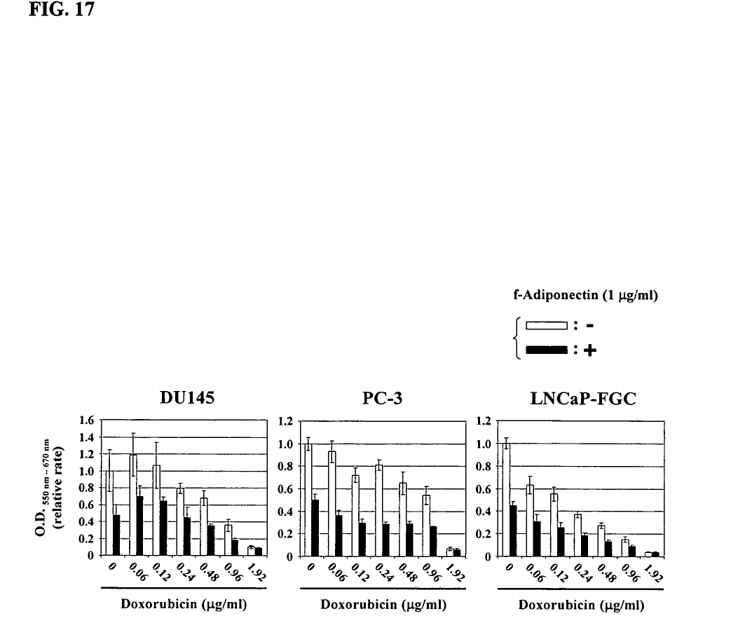

To determine whether f-adiponectin influences androgen-dependent prostate cancer cell growth, we performed the MTT assay with androgen-dependent LNCaP-FGC cells to look into the effect of f-adiponectin on DHT-stimulated cell growth. Intriguingly, 1 μg/ml f-adiponectin significantly suppressed DHT-stimulated cell growth (FIG. 16). Thus, f-adiponectin inhibits not only androgen-independent, but also androgen-dependent, prostate cancer cell growth.

f-Adiponectin Enhances Doxorubicin-Induced, Cell Growth Inhibition in Prostate cancer cells.

Doxorubicin is widely used for chemotherapy in a variety of cancers and inhibits prostate cancer cell growth (van Brussel, J. P., et al., Eur. J. Cancer 35:664-671, 1999; Tyagi, A. K., et al., Clin. Cancer Res. 8:3512-3519, 2002). We examined the effect of f-adiponectin on the doxorubicin inhibition of prostate cancer cell growth (FIG. 17). Consistent with a previous report (van Brussel, J. P., et al., supra, 1999), doxorubicin alone suppressed cell growth in a dose-dependent manner (0-1.92 μg/ml) in DU145, PC-3, and LNCaP-FGC cells. f-Adiponectin enhanced the doxorubicin suppression of prostate cancer cell growth at 1 μg/ml in these cell lines. This suggests that f-adiponectin may be useful for adjuvant therapy in combination with cytotoxic chemotherapy.

Discussion

Development of androgen independence by cancer cells is a fatal event during the natural history of prostate cancer. Thus, effective therapies and preventives need to be developed for AIPC. Prostate cancer, particularly its accelerated progression, is associated with obesity (adiposity) (Amling, C. L., et al., supra, 2001; Furuya, Y., et al., supra, 1998; Rodriguez, C., et al., supra, 2001). Adipocyte conditioned medium promotes prostate cancer cell growth, indicating that adipocytes secrete factors stimulatory to prostate cancer cell growth (Onuma, M., et al., supra, 2003). Adipose cytokines leptin, IGF-I and IL-6 are involved in metabolic syndrome (Kaaks, R. and Lukanova, supra, 2001; Wallenius, V., et al., supra, 2002; Zhang, Y., et al., Nature 372: 425-432, 1994), and their adipose production and blood levels rise in obesity (Maffei, M., et al., supra, 1995; Dagogo-Jack, S., et al., supra, 1996; Haffner, S. M., et al., supra, 1996; Ostlund, R. E., et al., supra, 1996; Frystyk, J., et al., supra, 1995; Nam, S. Y., et al., supra, 1997; Mohamed-Ali, V., et al., supra, 1997; Kern, P. A., et al., supra, 2001). Previous reports demonstrate that these cytokines stimulate AIPC cell growth (Onuma, M., et al., supra, 2003; Somasundar, P., et al., supra, 2004; Iwamura, M., et al., supra, 1993; Chung, T. D., et al., supra, 1999; Okamoto, M., et al., supra, 1997). Another adipose cytokine, adiponectin, decreases in circulation in obesity (Arita, Y., et al., supra, 1999; Cnop, M., et al., supra, 2003), and ameliorates obesity and diabetes mellitus (Fruebis, J., et al., supra, 2001; Berg, A. H., et al., supra, 2001). In this study, we identified adiponectin as a novel growth inhibitor in prostate cancer cell growth. We found f-adiponectin to inhibit AIPC cell growth drastically at subphysiological concentrations (FIG. 12) and the HMW form to be the inhibitory oligomeric form (FIG. 13). Therefore, the HMW form of f-adiponectin could be therapeutic for AIPC. Interestingly, f-adiponectin suppressed adipocyte conditioned medium-induced, prostate cancer cell growth (FIG. 14), as well as leptin- and/or IGF-I-stimulated, AIPC cell growth (FIG. 15). This strongly indicates that f-adiponectin competes with stimulatory adipose cytokines, such as leptin and IGF-I, to inhibit AIPC cell growth. Therefore, the balance of stimulatory adipose cytokine and adiponectin levels may be a determinant of prostate cancer prognosis: an increase in stimulatory cytokines with a concomitant decrease of adiponectin, especially its HMW form, in obesity may promote AIPC cell growth and accelerate hormone-resistant progression of the disease. This hypothesis is partially supported by a previous report showing that blood levels of IL-6 are associated with clinically evident hormone-resistant prostate cancer (Drachenberg, D. E., et al., supra, 1999). Thus, reducing obesity through diet and exercise, which increases blood f-adiponectin levels, may slow AIPC progression, and profiling blood adipose cytokine levels could be useful to predict AIPC prognosis.

We have recently reported that adiponectin inhibits constitutive STAT3 activation in androgen-independent DU145 cells (Miyazaki, T., et al., supra, 2005). Constitutive STAT3 activation is crucial in DU145 cell growth (Mora, L. B., et al., Cancer Res. 62:6659-6666, 2002). Therefore, STAT3 inhibition may mediate the adiponectin inhibition of AIPC cell growth when STAT3 is constitutively activated in cancer cells.

In addition to AIPC cells, f-adiponectin inhibited cell growth in androgen-dependent LNCaP-FGC cells (FIG. 12). Surprisingly, f-adiponectin suppressed DHT-stimulated, androgen-dependent cell growth drastically (FIG. 16). Therefore, f-adiponectin may be therapeutic and preventive for androgen-dependent prostate cancer, too: reducing obesity may inhibit progression of the androgen-dependent disease. Moreover, f-adiponectin could be used as adjuvant therapy to enhance the effect of androgen ablation on androgen-dependent prostate cancer. Furthermore, circulating f-adiponectin levels may predict prognosis of androgen-dependent prostate cancer, as well as AIPC.

Doxorubicin is a cytotoxic chemotherapy agent that has a broad spectrum of therapeutic activity against various types of cancers including prostate cancer (Raghavan, D., et al., Eur. J. Cancer 33:566-574, 1997; Heidenreich, A., et al., Cancer 101:948-956, 2004). Doxorubicin inhibits prostate cancer cell growth remarkably in both androgen-independent and -dependent cancer cells in vitro (van Brussel, J. P., et al., supra, 1999; Tyagi, A. K., et al., supra, 2002). However, doxorubicin chemotherapy is not very successful with prostate cancer patients as sufficient concentrations of the agent causes serious systemic toxicity (Von Hoff, D. D., et al., Ann. Intern. Med. 91:710-717, 1979). Therefore, specific delivery of doxorubicin to cancer cells (Heidenreich, A., et al., supra, 2004; Schally, A. V. and Nagy, A., Trends Endocrinol. Metab. 15:300-310, 2004) or lowering effective doses through adjuvant therapy is required for success in doxorubicin chemotherapy. We showed that f-adiponectin significantly enhanced doxorubicin-induced cell growth inhibition in prostate cancer cells (FIG. 17). Therefore, f-adiponectin may be useful for adjuvant therapy in combination with such existing therapeutic interventions as anti-cancer drugs, including doxorubicin, and irradiation. Furthermore, stimulation of f-adiponectin production through controlling obesity may also improve prostate cancer patients' responsiveness to such conventional therapies.

In conclusion, adiponectin is a molecular mediator between prostate cancer and obesity, and a common inhibitor for these two diseases. f-Adiponectin is inhibitory to both androgen-independent and -dependent prostate cancer. Therefore, f-adiponectin, as well as reducing obesity, could be therapeutic and preventive for both stages of prostate cancer. Furthermore, blood levels of adiponectin may help predict prostate cancer prognosis.

I claim:

1. A method of treating prostate cancer in a prostate cancer patient, comprising the step of decreasing or blocking the patient's leptin function or increasing adiponectin function

2. The method of claim 1 additionally comprising the step of decreasing or blocking the patient's IGF or IL-6 function.

3. The method of claim 1 wherein siRNA is used to decrease leptin interaction.

4. The method of claim 3 wherein the siRNA molecule is selected from the group of SEQ ID NO:1 and SEQ ID NO:2.

5. The method of claim 1 wherein antibodies are used to decrease leptin interaction.

6. The method of claim 1 wherein analogous leptin peptides are used to decrease leptin and/or adiponectin function.

7. The method of claim 6 wherein the peptides are selected from the group consisting of SEQ ID NOs:3-8.

8. The method of claim 1 wherein control of leptin function is through modification of the patient's body fat.

9. The method of claim 1 wherein the increase in adiponectin function is accomplished by administration of native or recombinant adiponectin or a nucleic acid sequence encoding adiponectin.

10. The method of claim 1 wherein increase in adiponectin function is via antibodies or small molecules that stimulate adiponectin receptors.

11. The method of claim 1 wherein native or recombinant adiponectin or a nucleic acid sequence encoding adiponectin is administered systemically.

12. The method of claim 1 wherein native or recombinant adiponectin or a nucleic acid sequence encoding adiponectin is administered directly to tumors or tissue surrounding a tumor.

13. A method of diagnosing a patient's prostate cancer risk comprising the step of examining the patient's blood concentration profiles of at least one adipose cytokines and correlating the level with prostate cancer diagnosis.

14. The method of claim 13 wherein the adipose cytokine is selected from the group consisting of leptin, adiponectin IGF-I, IGF-II, IL-6, TNF-α. PSA and DHT.

15. The method of claim 14 wherein multiple cytokines are examined.

16. The method of claim 15 wherein one of the selected cytokines is leptin.

17. The method of claim 15 wherein one of the selected cytokines is adiponectin.


SEQUENCE LISTING (23 sequences)

SEQ ID NO: 1 - RNA, 21 nt, Homo sapiens
aaagucaccgguuuggacuuc

SEQ ID NO: 2 - RNA, 21 nt, Homo sapiens
aagauggaccagacacuggca

SEQ ID NO: 3 - protein, 14 aa, Artificial
  Synthetic peptide.
EVVALSRLQGSLQD

SEQ ID NO: 4 - protein, 14 aa, Artificial
  Synthetic peptide.
EVVALDRLQGSLQD

SEQ ID NO: 5 - protein, 14 aa, Artificial
  Synthetic peptide.
EVVALSQLQGSLQD

SEQ ID NO: 6 - protein, 14 aa, Artificial
  Synthetic peptide.
EVVALDQLQGSLQD

SEQ ID NO: 7 - protein, 12 aa, Artificial
  Synthetic peptide.
KQKVTGLDFIPG

SEQ ID NO: 8 - protein, 12 aa, Artificial
  Synthetic peptide.
KQKVTGLNFIPG

SEQ ID NO: 9 - DNA, 24 bp, Artificial
  Synthetic oligonucleotide.
ttgtgccagtaattatttcctctt

SEQ ID NO: 10 - DNA, 18 bp, Artificial
  Synthetic oligonucleotide.
ctgtggccttccgcagtg

SEQ ID NO: 11 - DNA, 21 bp, Artificial
  Synthetic oligonucleotide.
acctccacccagtagttcctt

SEQ ID NO: 12 - DNA, 20 bp, Artificial
  Synthetic oligonucleotide.
agttggcacattgggttcat

SEQ ID NO: 13 - DNA, 20 bp, Artificial
  Synthetic oligonucleotide.
ctgatcagcgtggcgtattt

SEQ ID NO: 14 - DNA, 26 bp, Artificial
  Synthetic oligonucleotide.
tgaaggtcggtgtcaacggatttggc

SEQ ID NO: 15 - DNA, 24 bp, Artificial
  Synthetic oligonucleotide.
catgtaggccatgaggtccaccac

SEQ ID NO: 16 - DNA, 21 bp, Artificial
  Synthetic oligonucleotide.
aggacaacgactatctgctac

SEQ ID NO: 17 - DNA, 21 bp, Artificial
  Synthetic oligonucleotide.
catcccaaaaaccaccttctc

SEQ ID NO: 18 - DNA, 20 bp, Artificial
  Synthetic oligonucleotide.
agagaaagtggtggggaaag

SEQ ID NO: 19 - DNA, 20 bp, Artificial
  Synthetic oligonucleotide.
gggcgagggaggaaaataac

SEQ ID NO: 20 - DNA, 24 bp, Artificial
  Synthetic oligonucleotide.
aattccatttcccgtaaatccctg

SEQ ID NO: 21 - DNA, 24 bp, Artificial
  Synthetic oligonucleotide.
aattcagggatttacgggaaatgg

SEQ ID NO: 22 - RNA, 70 nt, Homo sapiens
aggaaucgcagcgccaacgguugcaaggcccaagaagcccauccugggaaggaaaaugcauuggggaacc

SEQ ID NO: 23 - RNA, 90 nt, Homo sapiens
cuccaaacagaaagucaccgguuuggacuucauuccugggcuccaccccauccugaccuuauccaagauggaccagacacuggcagucua